(19) United States
(12) Patent Application Publication
(10) Pub. No.: US 2024/0049943 A1
(43) Pub. Date: Feb. 15, 2024

(54) FLUORESCENCE EVALUATION APPARATUSES, SYSTEMS, AND METHODS

(71) Applicant: Intuitive Surgical Operations, Inc., Sunnyvale, CA (US)

(72) Inventors: Dennise D. Dalma-Weiszhausz, Palo Alto, CA (US); Ian E. McDowall, Woodside, CA (US); Theodore W. Rogers, Alameda, CA (US)

(21) Appl. No.: 18/266,770

(22) PCT Filed: Dec. 30, 2021
(86) PCT No.: PCT/US2021/065600

§ 371 Date: Jun. 12, 2023

Related U.S. Application Data
(60) Provisional application No. 63/132,684, filed on Dec. 31, 2020.

(51) Int. Cl. A61B 1/00; A61B 1/04; A61B 1/06

(52) U.S. Cl. CPC A61B 1/000095; A61B 1/043; A61B 1/0646; A61B 1/0638; A61B 1/00057

(57) ABSTRACT

A fluorescence evaluation apparatus includes a substrate configured to be inserted into a body through a channel having an inside diameter between about 5 millimeters (mm) and about 30 mm and a plurality of fluorescence swatches of a fluorescence imaging agent, each fluorescence swatch being arranged on the substrate and having a different concentration of the fluorescence imaging agent.

### RELATED APPLICATIONS

The present application claims priority to U.S. Provisional Patent Application No. 63/132,684, filed Dec. 31, 2020, the contents of which are hereby incorporated by reference in their entirety.

### BACKGROUND INFORMATION

An imaging device (e.g., an endoscope) may be used during a surgical procedure to capture images of a surgical area associated with a patient. The images may be presented (e.g., in the form of a video stream) during the surgical procedure to assist the surgeon in performing the surgical procedure. In some scenarios, the images of the surgical area may include or be augmented with other captured images, such as fluorescence images. Fluorescence images are generated based on detected fluorescence emitted by a fluorescence imaging agent upon excitation by a light source. The fluorescence images may be used, for example, to highlight certain portions, tissues, or tissue perfusion of the surgical area in a selected color (e.g., green). However, the fluorescence images do not provide a measure of the concentration of the fluorescence imaging agent, information which may be useful intraoperatively as well as pre-operatively (e.g., for calibration of the imaging device).

### SUMMARY

The following description presents a simplified summary of one or more aspects of the methods and systems described herein in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects of the methods and systems described herein in a simplified form as a prelude to the more detailed description that is presented below.

An illustrative system may comprise: a fluorescence evaluation apparatus comprising a substrate and a plurality of fluorescence swatches of a fluorescence imaging agent, each fluorescence swatch being arranged on the substrate and having a different concentration of the fluorescence imaging agent; an imaging device configured to illuminate a scene within a body with fluorescence excitation illumination, the scene including the fluorescence evaluation apparatus and tissue, detect fluorescence emitted from the plurality of fluorescence swatches, and detect fluorescence emitted from a region of the tissue; and a display device configured to display an image of the scene, the image showing the fluorescence emitted from the plurality of fluorescence swatches and the fluorescence emitted from the region of tissue.

An illustrative fluorescence evaluation apparatus may comprise a substrate configured to be inserted into a body through a channel having an inside diameter between about 5 millimeters (mm) and about 30 mm; and a plurality of fluorescence swatches of a fluorescence imaging agent, each fluorescence swatch being arranged on the substrate and having a different concentration of the fluorescence imaging agent.

An illustrative method may comprise: forming a substrate configured to be inserted into a body through a channel having an inside diameter between about 5 millimeters (mm) and about 30 mm; and arranging, on the substrate, a plurality of fluorescence swatches of a fluorescence imaging agent, each fluorescence swatch having a different concentration of the fluorescence imaging agent.

An illustrative method may comprise: illuminating, by an imaging device, a scene within a body with fluorescence excitation illumination, the scene including a fluorescence evaluation apparatus and tissue, the fluorescence evaluation apparatus comprising a substrate and a plurality of fluorescence swatches of a fluorescence imaging agent, each fluorescence swatch being arranged on the substrate and having a different concentration of the fluorescence imaging agent; detecting, by the imaging device, fluorescence emitted from the plurality of fluorescence swatches; detecting, by the imaging device, fluorescence emitted from a region of the tissue; and providing, by the imaging device for display on a display device, image data representative of the scene, the image showing the fluorescence emitted from the plurality of fluorescence swatches and the fluorescence emitted from the region of tissue.

An illustrative method may comprise: administering a fluorescence imaging agent to a body; positioning a fluorescence evaluation apparatus at a scene within the body and associated with a medical procedure, the fluorescence evaluation apparatus comprising a substrate and a plurality of fluorescence swatches of an equivalent of the fluorescence imaging agent, each fluorescence swatch being arranged on the substrate and having a different concentration of the equivalent of the fluorescence imaging agent; and illuminating the scene with fluorescence excitation illumination while the fluorescence evaluation apparatus is positioned at the scene.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIGS. 1A to 5B show various illustrative configurations of a fluorescence evaluation apparatus.

FIGS. 6 to 11 show various illustrative methods of making a fluorescence evaluation apparatus.

FIG. 12 shows a functional diagram of an illustrative imaging system that may capture fluorescence images of a scene including a fluorescence evaluation apparatus.

FIG. 13 shows an illustrative augmented image captured by an imaging device and depicting a scene comprising tissue and a fluorescence evaluation apparatus and that may be used for in situ assessment of the concentration of a fluorescence imaging agent in the tissue.

FIG. 14 shows another illustrative augmented image of a scene captured by an imaging device and depicting a scene comprising tissue and a fluorescence evaluation apparatus that may be used for in vivo assessment of tissue perfusion.

FIG. 15 shows an illustrative fluorescence evaluation system that configured to evaluate fluorescence emitted from a scene with the aid of a fluorescence evaluation apparatus.

FIG. 16 shows an illustrative computer-assisted surgical system.

FIG. 17 shows an illustrative method.

FIG. 18 shows an illustrative computing device.

### DETAILED DESCRIPTION

Fluorescence evaluation apparatuses, systems, and methods are described herein. Various specific embodiments will be described in detail with reference to the figures. It will be understood that the specific embodiments described below are provided as non-limiting examples of how various novel and inventive principles may be applied in various situations. Additionally, it will be understood that other examples not explicitly described herein may also be captured by the scope of the claims set forth below. Systems and methods described herein may provide one or more benefits that will be explicitly described or made apparent below.

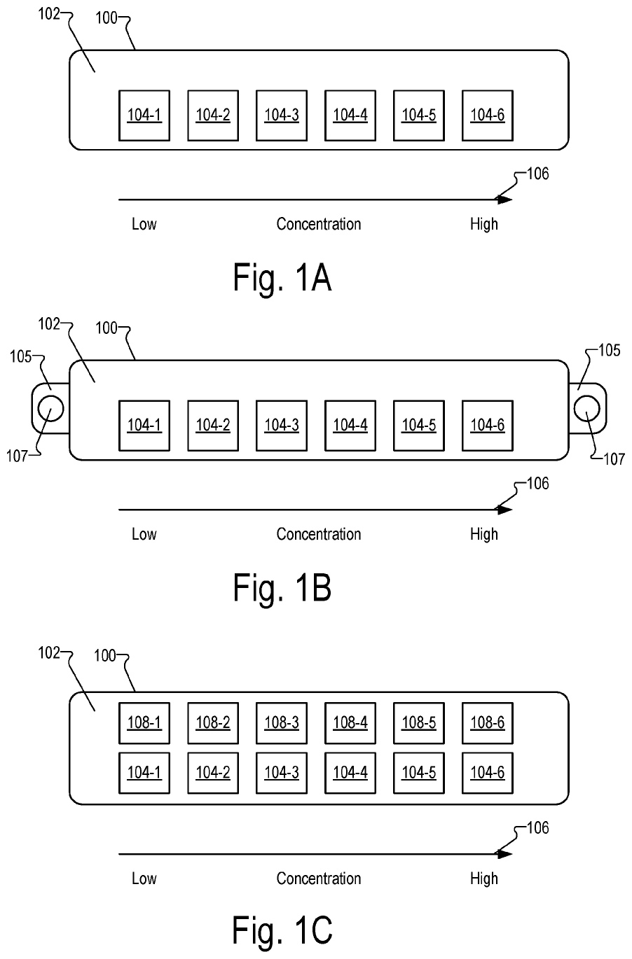

FIG. 1A shows an illustrative configuration of a fluorescence evaluation apparatus 100. As shown, fluorescence evaluation apparatus 100 includes a substrate 102 and a plurality of fluorescence swatches 104 (e.g., fluorescence swatches 104-1 through 104-6) of a fluorescence imaging agent arranged on substrate 102.

Substrate 102 may be formed of any suitable solid material, including polymers, ceramics, composites, metals, or a combination thereof. Suitable polymers may include, without limitation, elastomers (e.g., silicone rubbers, natural rubbers, fluoroelastomers (such as polytetrafluoroethylene (PTFE), perfluoroether (PFA), fluorinated ethylene propylene (FEP)), ethylene propylene diene monomer (EPDM) rubbers, nitrile rubbers (e.g., acrylonitrile-butadiene rubber), and polyolefin elastomers), synthetic polymers (e.g., epoxy, resins, polyvinylchloride (PVC), polyethylene (PE), polyethylene glycol (PEG), polypropylene (PP), polymethylmethacrylate (PMMA), polystyrene (PS), polyurethanes (PU), polyamides, polyethyleneterephthalate (PET), glycol-modified PET, polysulfone, polyetherimide (PEI), polyethersulfone (PES), polyarylsulfone, polyetheretherketone (PEEK), polycarbonates, ethylene vinyl acetate (EVA), styrene butadiene copolymer (SBC), polylactic acid (PLA), polyglycolic acid (PGA), polycaprolactone (PCL), acrylics, acrylonitrile butadiene styrene (ABS), and cellulose acetate butyrate (CAB)), and natural polymers (e.g., rubber, carbohydrate polymers such as a polysaccharide (e.g., hyaluronic acid, chitosan, etc.), lipid polymers (e.g., triglycerides, triacylglycerols, triacylglycerides, phospholipids, waxes, etc.), and protein polymers (e.g., collagen, fibrin), etc.).

Substrate 102 may be a single layer (e.g., a single material layer). In alternative examples, substrate 102 may be a lamination of multiple layers, which may be made of the same or different materials. As will be explained below in more detail, a laminated substrate 102 may help prevent degradation of fluorescence swatches 104, such as by preventing a fluorescence imaging agent in fluorescence swatches 104 from leaching out of substrate 102.

Substrate 102 may be formed of a material that is transparent to visible light and/or near-infrared (NIR) light. For example, substrate 102 may be transparent to visible light to enable a user to view a scene located behind fluorescence evaluation apparatus 100. Additionally or alternatively, substrate 102 may be transparent to NIR light having a wavelength between about 800 nanometers (nm) and about 850 to thereby enable an imaging sensor to capture fluorescence emitted by a fluorescence imaging agent present in a subject (e.g., tissue) located behind fluorescence evaluation apparatus 100. In yet further examples, substrate 102 may be opaque for visible light and/or NIR light.

In some examples, substrate 102 is configured to be inserted into a body through a tubular channel, such as a cannula, a channel created by surgical incision, or a natural channel of the body (e.g., the intestines or the esophagus). For instance, a surgical instrument (e.g., a surgical instrument connected to a manipulator arm of a robotic medical system) may hold fluorescence evaluation apparatus 100 (e.g., by grasping substrate 102) and may insert fluorescence evaluation apparatus 100 into the body by way of a cannula or a natural channel of the body. The channel may be curved or straight.

Substrate 102 may be configured to be inserted into a body through a tubular channel in any suitable way. In some examples, substrate 102 has a small form factor such that fluorescence evaluation apparatus 100 may fit through a small channel, such as a channel having an inside diameter between about 5 mm and about 30 mm. In further examples, the channel may have an inside diameter between about 5 mm and about 12 mm. In yet further examples, the channel may have an inside diameter between about 5 mm and about 8 mm. In some examples, the channel comprises a cannula having an inside diameter of about 5 mm, 8 mm, 10 mm, or 12 mm, and substrate 102 has a width and a thickness that is less than an inside diameter of the channel. For example, a substrate 102 configured to fit through a cannula having an 8 mm inside diameter may have a thickness of about 2 mm to about 5 mm and a width of about 6.25 mm to about 7.75 mm. A substrate 102 configured to fit through a cannula having a 10 mm inside diameter may have a thickness of about 2 mm to about 7 mm and a width of about 7 mm to about 9.8 mm. A substrate 102 configured to fit through a cannula having a 12 mm inside diameter may have a thickness of about 2 mm to about 8 mm and a width of about 8.9 mm to about 11.8 mm. Substrate 102 may have any suitable length. For example, substrate 102 may be about 10 cm long or less. In some examples, substrate 102 may be about 8 cm long or less. It will be recognized that the foregoing dimensions are only illustrative and not limiting.

In some embodiments, such as when substrate 102 may be wider than an inside diameter of the channel and/or when the channel is curved, substrate 102 may be flexible and configured to bend or twist as necessary to fit through the channel. For example, substrate 102 may be configured to bend latitudinally (across a width of substrate 102) and/or longitudinally (along a length of substrate 102) so that fluorescence evaluation apparatus 100 may be inserted through a straight or curved channel having a small inside diameter. In additional or alternative examples, substrate 102 may be wider than the inside diameter of the channel but may be configured to fold to a width that is smaller than the inside diameter of the channel. In such embodiment, substrate 102 may be formed of two or more substrates connected to one another by one or more movable connections (e.g., hinge joints).

Substrate 102 may have any suitable shape. In some examples, as shown in FIG. 1A, substrate 102 is rectangular and elongate. However, substrate 102 may have any other suitable shape, such as circular, elliptical, cylindrical, triangular, or a freeform shape. The corners of substrate 102 may be rounded so fluorescence evaluation apparatus 100 may be used near patient tissue. In some examples, substrate 102 is substantially flat. In alternative examples, substrate 102 is curved latitudinally. The curvature may facilitate insertion of substrate 102 through a cannula. For example, a radius of curvature of substrate 102 may be similar to a radius of curvature of the cannula through which substrate 102 may be inserted. The curvature may also prevent longitudinal bending of substrate 102 and thus help maintain structural integrity of fluorescence evaluation apparatus 100 during use.

As mentioned, a plurality of fluorescence swatches 104 of a fluorescence imaging agent are arranged on substrate 102. While FIG. 1A shows that fluorescence evaluation apparatus 100 has six fluorescence swatches 104, fluorescence evaluation apparatus 100 may have more or fewer fluorescence swatches as may serve a particular implementation.

Each fluorescence swatch 104 comprises a distinct region having a different concentration of the fluorescence imaging agent. The fluorescence imaging agent may be any molecule, mixture, or complex configured to emit fluorescence when excited with fluorescence excitation illumination. In some examples, the fluorescence imaging agent comprises, or is included in, a fluorescent coating material configured for near-infrared coating of equipment (NICE). Suitable fluorescence imaging agents may include, for example, biologically endogenous compounds (e.g., flavin adenine dinucleotide (FAD), reduced nicotinamide adenine dinucleotide (NADH), riboflavin, collagen, etc.) as well as biologically exogenous compounds such as molecular dyes, organic dyes, proteins, IR-125, indocyanine green (ICG), fluorescein, rhodamine, quantum dots, organometallic complexes, lanthanides, fullerenes, nanotubes, nanoparticles, up-conversion materials, and the like.

In some examples, the fluorescence imaging agent may be combined with or in a support matrix (e.g., dissolved in a solvent), such as a biocompatible polymer (e.g., poly(methyl methacrylate) (PMMA), a polyurethane polymer, or any other suitable polymer), to produce a fluorescent mixture. The fluorescent mixture may also include other components, such as an absorbing agent (e.g., hemin) and/or a scattering agent (e.g., titanium oxide (TiO₂). In some examples, the fluorescence imaging agent is substantially resistant to photobleaching. Illustrative fluorescence imaging agents, and methods of making them, are described in Ruiz et al., “Indocyanine green matching phantom for fluorescence-guided surgery imaging system characterization and performance assessment,” J. Biomed. Opt. 25(5), 056003 (2020). When used in the singular; fluorescence imaging agent refers to a particular type of fluorescence imaging agent (e.g., ICG, fluorescein, rhodamine, etc.), whether as a single molecule or a population of molecules.

In some examples, fluorescence evaluation apparatus 100 may be used for assessing fluorescence emitted by a fluorescence imaging agent (e.g., ICG) administered to a body and that is present in tissue. Accordingly, the fluorescence imaging agent used in fluorescence swatches 104 may comprise an equivalent of the fluorescence imaging agent administered to the body. An equivalent of the fluorescence imaging agent may be the same fluorescence imaging agent or a fluorescence imaging agent that behaves similarly to the fluorescence imaging agent administered to the body. An equivalent fluorescence imaging agent may behave similarly to a fluorescence imaging agent present in tissue when the equivalent fluorescence imaging agent has a substantially similar response to fluorescence excitation illumination (e.g., is excited by substantially the same wavelength or wavelength band of fluorescence excitation illumination and/or emits fluorescence in substantially same wavelength or wavelength band and/or with substantially the same intensity).

For example, when ICG is administered to a patient, the equivalent fluorescence imaging agent included in fluorescence evaluation apparatus 100 may include ICG and/or any other fluorescence imaging agent that behaves similarly to ICG, such as IR-125. For example, the fluorescence imaging agent in fluorescence swatches 104 may be configured to be excited by fluorescence excitation illumination having a wavelength between about 750 nm and about 810 nm and may be configured to emit, in response to excitation by the fluorescence excitation illumination, fluorescence having a peak intensity at a wavelength between about 800 nm to about 850 nm. In further examples, the fluorescence imaging agent may be configured to be excited by fluorescence excitation illumination having a wavelength between about 780 nm and about 810 nm. In yet further examples, the fluorescence imaging agent may be configured to be excited by fluorescence excitation illumination having a wavelength of about 785 nm and/or about 805 nm. In further examples, the fluorescence imaging agent may be configured to emit, in response to excitation by the fluorescence excitation illumination, fluorescence having a peak intensity at a wavelength between about 810 nm to about 840 nm. In yet further examples, the fluorescence imaging agent may be configured to emit, in response to excitation by the fluorescence excitation illumination, fluorescence having a peak intensity of about 830 nm.

In further examples, substrate 102 may include one or more narrow bandpass filters configured to filter fluorescence excitation illumination to between about 750 nm and about 810 and/or filter emitted fluorescence to between about 800 nm and about 850 nm. The fluorescence imaging agent and/or narrow bandpass filters may alternatively configure for any other wavelength range of fluorescence excitation illumination and/or emitted fluorescence as may serve a particular implementation. The narrow bandpass filters may be arranged as a separate layer of substrate 102.

Fluorescence swatches 104 may be arranged on substrate 102 in any suitable configuration. In some examples, as indicated by concentration legend 106, fluorescence swatches 104 are arranged on substrate 102 in a single line in order of increasing concentration of the fluorescence imaging agent. For instance, fluorescence swatch 104-1 has the lowest concentration of the fluorescence imaging agent, and the concentration of fluorescence swatches 104-2 through 104-6 progressively increases to fluorescence swatch 104-6, which has the highest concentration of the fluorescence imaging agent. In some examples, the concentration of the fluorescence imaging agent in fluorescence swatches 104 varies stepwise from low to high in equal steps. In other examples, the concentration may vary in unequal steps. In yet further embodiments, fluorescence swatches 104 are not arranged in order of increasing concentration, but are arranged randomly or in any other suitable order, such as to maximize contrast between adjacent fluorescence swatches 104. Furthermore, in other embodiments fluorescence swatches 104 may be arranged in more than one line.

In some examples, fluorescence swatches 104 are arranged along or near an edge of substrate 102 so that fluorescence swatches 104 can be easily compared with fluorescence emitted from a subject (e.g., patient tissue). In alternative examples, such as when substrate 102 is transparent to NIR and/or visible light, fluorescence swatches 104 may be arranged away from an edge of substrate 102 (e.g., in the center of substrate 102).

FIG. 1A shows that adjacent fluorescence swatches 104 are separated from one another by a gap. In alternative configurations, adjacent fluorescence swatches 104 are in contact with one another. Consequently, fluorescence emitted by fluorescence swatches 104 may appear as a continuous spectrum.

Fluorescence swatches 104 may be arranged on substrate 102 in any suitable way. In some examples, fluorescence swatches 104 are arranged on substrate 102 by embedding, dissolving, or doping the fluorescence imaging agent in different concentrations in distinct regions of substrate 102. For example, substrate 102 may be doped with different concentrations of the fluorescence imaging agent in distinct regions of substrate 102. In alternative examples, the fluorescence imaging agent may be coated on substrate 102, adhered to substrate 102, absorbed into substrate 102, laminated as a separate layer on substrate 102, or any other suitable method. For example, each fluorescence swatch 104 may be formed separately from substrate 102 and may be attached to a surface of substrate 102 or positioned in a recessed well formed in substrate 102. For instance, fluorescence swatches 104 may be formed as individual solid “tiles” of a fluorescent mixture. The fluorescent mixture may be formed by dissolving the fluorescence imaging agent in a liquid polymer or other solvent to form the fluorescent mixture. For example, the fluorescence imaging agent may be ICG and the solvent may be any suitable polymer, such as a polyurethane polymer, in which ICG may dissolve to form a homogeneous mixture. The fluorescent mixture may then be 3D printed or injection molded into the desired tile shape and cured to form solid tiles. The solid tiles may then be secured in recessed wells in substrate 102, such as by friction, an adhesive, or a mechanical fastener (e.g., a screw, snap fit, a cover layer on substrate 102, etc.). In other examples, recessed wells may be formed in substrate 102 and a fluorescent mixture may be deposited in the wells and cured within the wells. In yet further examples, fluorescence swatches 104 are not arranged on a common substrate but instead are arranged on separate substrates that are joined or held together (temporarily or permanently) to form a single fluorescence evaluation apparatus 100. The separate substrates may be held together rigidly or flexibly and may be held together in any suitable way, such as by an adhesive, an outer band or ring, a casing, hinges, joints, or fasteners.

As mentioned above, in some examples the fluorescence imaging agent may be arranged on a surface of a first layer of substrate 102, and a second layer of substrate 102 may be laminated on top of the first layer and the fluorescence imaging agent. In this way, the fluorescence imaging agent may be held between two layers of substrate 102, which may help reduce or prevent degradation of the fluorescence imaging agent, such as by preventing leaching of the fluorescence imaging agent from substrate 102. The first layer and second layers of substrate 102 may be made of the same or different materials. In some examples, as will be described below, the first layer may be formed of a material configured to quench visible light reflected from fluorescence swatches 104.

In some examples, the fluorescence imaging agent may be encapsulated in an encapsulating agent that is configured to reduce or prevent photobleaching of the fluorescence imaging agent and/or leaching of the fluorescence imaging agent out of substrate 102. For example, the encapsulating agent may be a surfactant, a polymer, or a dispersing agent that is then embedded in substrate 102 or added to a solution or a support matrix. Suitable encapsulating agents may include, for example, carbohydrate polymers (e.g., polysaccharides), lipid polymers (e.g., triglycerides, triacylglycerols, triacylglycerides, phospholipids, waxes, etc.), steroids (e.g., cholesterol), and organometallic materials.

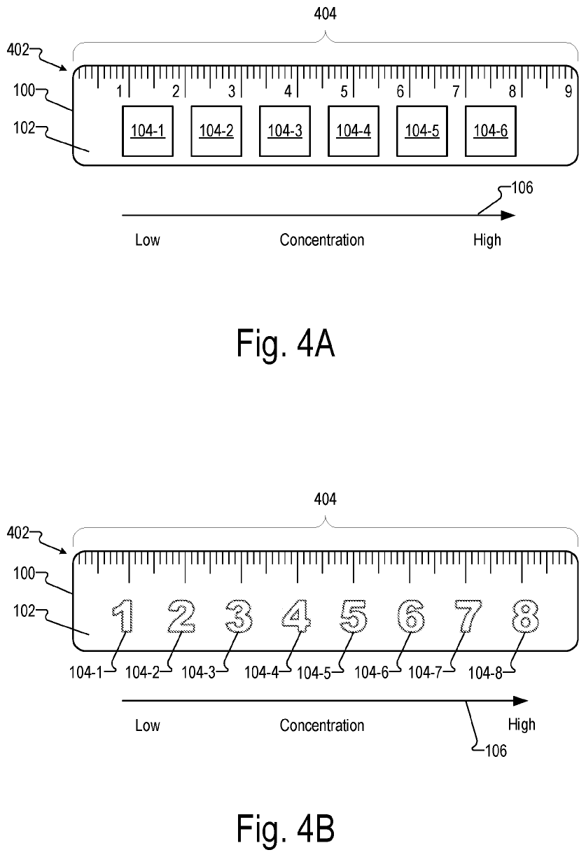

Fluorescence swatches 104 may have any suitable shape and size. As shown in FIG. 1A, fluorescence swatches 104 are square and have the same size. In alternative embodiments, fluorescence swatches 104 may have any other shape or shapes, such as rectangular, circular, elliptical, triangular, polygonal, freeform, etc. In some examples, each fluorescence swatch 104 has a different shape to allow a user to easily distinguish fluorescence swatches 104 from one another. For example, the shapes of fluorescence swatches 104 may be block lettering or numbering (e.g., “1” to “6”, see FIG. 4B). In this way, a user can easily remember which fluorescence swatch 104 emits fluorescence that matches fluorescence emitted from a scene. In yet further examples, each fluorescence swatch 104 may have a unique shape that may be detected and used to automate some of the processes described herein.

As explained above, fluorescence swatches 104 emit fluorescence when fluorescence evaluation apparatus 100 is illuminated with fluorescence excitation illumination. An imaging device may capture the emitted fluorescence, and an imaging system may use the captured fluorescence to generate and display a fluorescence image. In some scenarios, the fluorescence evaluation apparatus 100 may simultaneously be illuminated with visible light. The imaging device may capture the reflected visible light, which may be used to generate a visible light image that is displayed with the fluorescence image (e.g., as an augmented image). However, specular reflection of high intensity visible light may obscure the fluorescence emitted from fluorescence swatches 104 and saturate the displayed image so that the fluorescence image is not sufficiently visible. To address these issues, fluorescence evaluation apparatus 100 may be configured to quench (at least partially) the specular reflection of visible light from fluorescence swatches 104. The specular reflection of visible light may be quenched in any suitable way.

In some examples, fluorescence swatches 104 may be formed with surface irregularities (surface roughness) so that visible light is primarily diffusely reflected from fluorescence swatches 104. The surface irregularities may be formed in any suitable way, such as by sanding, sand blasting, scratching, etching, or any other mechanical or chemical process. Diffuse reflection of visible light from fluorescence swatches 104 reduces the intensity of reflected visible light, thereby preventing saturation of the displayed image.

Additionally or alternatively, fluorescence swatches 104 may be formed of a fluorescent mixture having a color that reduces the intensity of reflection of visible light. In some examples, the fluorescent mixture includes a base material (e.g., a biocompatible polymer) that, when cured, is transparent, translucent, and/or light-absorbing for all or parts of the visible spectrum. In other examples, the base material is non-white, e.g., gray, black, or any other suitable color. A gray base material may be obtained, for example, by mixing a white resin and a black resin to form the base material. In some examples, the ratio of the white resin to the black resin ranges from approximately 95%/5% to approximately 75/25% by weight, although any other suitable ratio may be used as may serve a particular implementation.

In some examples, the fluorescent mixture used to form fluorescence swatches 104 may include one or more additives configured to quench specular reflection of visible light. For example, the fluorescent mixture may include an optical dust obtained from an optical filter material (e.g., a long-pass filter). The optical dust may be obtained by grinding or pulverizing (or other process) an optical filter material into a fine powder. The optical filter material is configured to filter out visible light or light having a wavelength shorter than a wavelength (e.g., a peak wavelength, an average wavelength, etc.) of fluorescence emitted by the fluorescence imaging agent. The optical dust may be mixed in with the fluorescent mixture prior to curing the fluorescent mixture. Additional or alternative additives may be included in the fluorescent mixture, such as an absorbing agent and/or a scattering agent.

Specular reflection of visible light from fluorescence swatches 104 may additionally or alternatively be quenched by a layer of optical filter material over fluorescence swatches 104. For example, a color gel (also referred to as a color filter), long-pass filter, or other transparent filter film may be provided on substrate 102 over fluorescence swatches 104. The optical filter material is configured to filter out portions (or all) of the reflected visible light or light having a wavelength shorter than a wavelength (e.g., a peak wavelength, an average wavelength, etc.) of fluorescence emitted by the fluorescence imaging agent. The layer of optical filter material may be formed over all of substrate 102 or only over fluorescence swatches 104 (e.g., in a top portion of recessed wells in which fluorescence swatches 104 are arranged).

FIG. 1B shows another illustrative configuration of fluorescence evaluation apparatus 100. FIG. 1B is similar to FIG. 1A except that, in FIG. 1B, substrate 102 includes tabs 105 protruding from substrate 102 at opposite ends of substrate 102. Tabs 105 may be formed integrally with substrate 102 or may be formed separately and attached to substrate 102. Tabs 105 may be used by a user or an instrument to grip fluorescence evaluation apparatus 100 without gripping fluorescence swatches 104. Tabs 105 include holes 107 that may be used to secure fluorescence evaluation apparatus in place, such as with sutures, as well as for using sutures or other devices to pull fluorescence evaluation apparatus from the subject. While FIG. 1B shows two tabs 105 and two holes 107, substrate 102 may include any other number of tabs 105 and holes 107, such as one or more than two. Additionally, holes 107 may be omitted from tabs 105 and/or may be provided elsewhere on substrate 102.

FIG. 1C shows another illustrative configuration of fluorescence evaluation apparatus 100. FIG. 1C is similar to FIG. 1A except that in FIG. 1C fluorescence evaluation apparatus includes a plurality of additional fluorescence swatches 108 (e.g., additional fluorescence swatches 108-1 through 108-6) of an additional fluorescence imaging agent arranged on substrate 102. The additional fluorescence imaging agent of additional fluorescence swatches 108 is different from the fluorescence imaging agent of fluorescence swatches 104. For example, fluorescence swatches 104 may comprise ICG and additional fluorescence swatches 108 may comprise IR-125. In some examples, the additional fluorescence imaging agent of additional fluorescence swatches 108 is configured to emit fluorescence having a peak wavelength that is different from a peak wavelength of fluorescence emitted by the fluorescence imaging agent of fluorescence swatches 104. Additionally or alternatively, the additional fluorescence imaging agent of additional fluorescence swatches 108 is configured to be excited by fluorescence excitation illumination having a first wavelength or wavelength range that is different from a wavelength or wavelength range of fluorescence excitation illumination configured to excite the fluorescence imaging agent of fluorescence swatches 104.

Additional fluorescence swatches 108 may be arranged and configured in any of the ways described herein with respect to fluorescence swatches 104. While FIG. 1C shows that fluorescence evaluation apparatus 100 has six additional fluorescence swatches 108, fluorescence evaluation apparatus 100 may have more or fewer additional fluorescence swatches as may serve a particular implementation. In some examples, the additional fluorescence imaging agent may be embedded in substrate 102 in the same regions as fluorescence swatches 104 rather than in regions separate from fluorescence swatches 104.

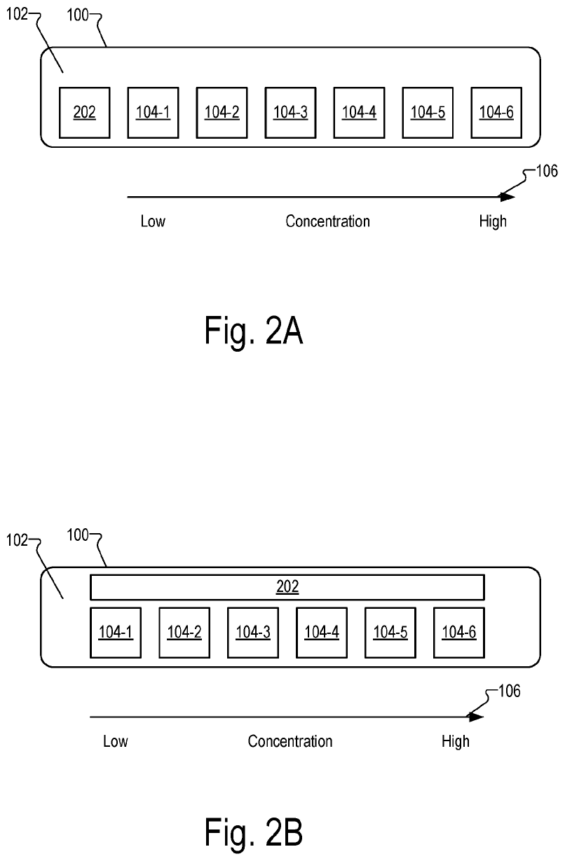

FIG. 2A shows another illustrative configuration of fluorescence evaluation apparatus 100. FIG. 2A is similar to FIG. 1A except that in FIG. 2A fluorescence evaluation apparatus 100 includes, in addition to fluorescence swatches 104, a reference fluorescence swatch 202 of the fluorescence imaging agent. Reference fluorescence swatch 202 is arranged on substrate 102 and has the same concentration of the fluorescence imaging agent as one of the fluorescence swatches 104 (e.g., fluorescence swatch 104-6). Reference fluorescence swatch 202 is arranged on substrate 102 away from the fluorescence swatch 104 having the same concentration. For example, reference fluorescence swatch 202 and fluorescence swatch 104-6 are arranged on opposite ends of substrate 102.

Reference fluorescence swatch 202 serves as a reference for comparison with the fluorescence swatch 104 having the same concentration. For example, when fluorescence evaluation apparatus 100 is illuminated with fluorescence excitation illumination from a fluorescence excitation illumination source (e.g., a distal end of an endoscope), fluorescence emitted by fluorescence swatch 104-6 and fluorescence emitted by reference fluorescence swatch 202 should have substantially the same intensity. If, however, reference fluorescence swatch 202 is positioned farther or closer to the fluorescence excitation illumination source than is fluorescence swatch 104-6, the intensity of the fluorescence emitted from fluorescence swatch 104-6 and reference fluorescence swatch 202 will be different. As a result, fluorescence swatches 104 farther from fluorescence swatch 104-6 might not be reliably used for evaluation of fluorescence emitted from the scene. Thus, a comparison of fluorescence swatch 104-6 with reference fluorescence swatch 202 may indicate when fluorescence evaluation apparatus 100 is not properly positioned within the scene. A user may adjust a position of fluorescence evaluation apparatus 100 (e.g., adjust a position of a surgical instrument holding fluorescence evaluation apparatus 100) until the intensity of the emitted fluorescence from fluorescence swatch 104-6 and the intensity of the emitted fluorescence from reference fluorescence swatch 202 are substantially the same.

Reference fluorescence swatch 202 may have any shape and configuration as may serve a particular implementation. In some examples, reference fluorescence swatch 202 has a shape (e.g., a triangle) that is different than a shape of the fluorescence swatches 104 (e.g., rectangular). In alternative examples in which fluorescence swatches 104 each have a different shape, a shape of reference fluorescence swatch 202 may be the same as the shape of the fluorescence swatch 104 having the same concentration.

FIG. 2B shows another illustrative configuration of reference fluorescence swatch 202. FIG. 2B is similar to FIG. 2A except that in FIG. 2B reference fluorescence swatch 202 extends along the plurality of fluorescence swatch 104. Accordingly, each fluorescence swatch 104 can be easily compared with reference fluorescence swatch 202. Reference fluorescence swatch 202 may have the same concentration of the fluorescence imaging agent as any one of fluorescence swatches 104.

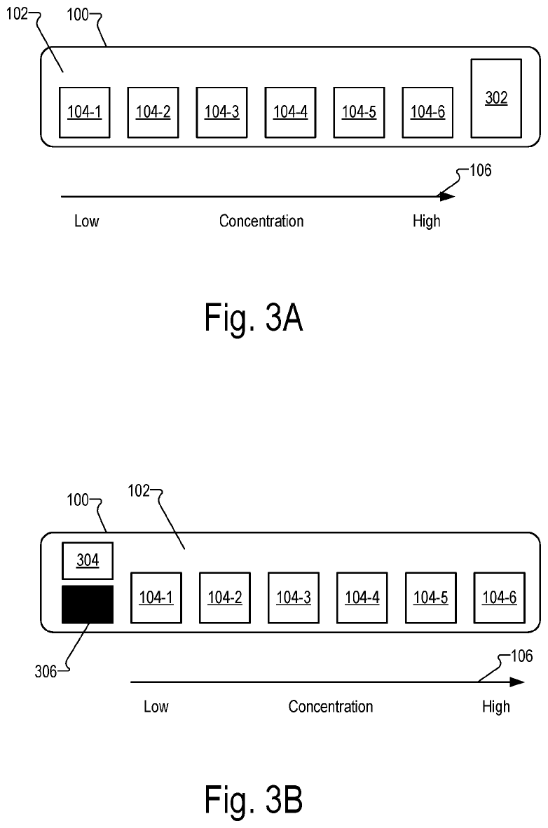

FIG. 3A shows another illustrative configuration of fluorescence evaluation apparatus 100. FIG. 3A is similar to FIG. 1A except that in FIG. 3A fluorescence evaluation apparatus 100 includes a NIR light swatch 302 arranged on substrate 102. NIR light swatch 302 comprises an up-converting agent configured to emit visible light (e.g., red light or green light) when excited by NIR light. Any suitable up-converting agent may be used, such as up-converting nanoparticles (e.g., lanthanide-doped nanoparticles, semiconductor nanoparticles, quantum dots, etc.). In some examples, the up-converting agent may be configured to be excited by NIR light having a wavelength between about 785 nm and about 980 nm and emit visible light. Accordingly, NIR light swatch 302 may visibly show when fluorescence evaluation apparatus 100 is illuminated with NIR fluorescence excitation illumination. In this way, the presence of NIR light and/or the functioning of a NIR light source may be easily confirmed.

FIG. 3B shows another illustrative configuration of fluorescence evaluation apparatus 100. FIG. 3B is similar to FIG. 1A except that in FIG. 3B fluorescence evaluation apparatus 100 includes a white surface swatch 304 and a black surface swatch 306 arranged on substrate 102. White surface swatch 304 is a pure white (or substantially pure white) surface and may include any material arranged on or in substrate 102. Black surface swatch 306 comprises a pure black (or substantially pure black) light-absorbing surface and may include any material arranged on substrate 102, such as Vantablack (Surrey NanoSystems, Newhaven, UK). White surface swatch 304 and black surface swatch 306 may be arranged on substrate 102 in any suitable way, such as by embedding, coating, absorbing, or adhering the material in or on substrate 102. White surface swatch 304 and black surface swatch 306 may be used for calibration of an imaging device (e.g., an endoscope). For example, a calibration system may measure reflectance from white surface swatch 304 and black surface swatch 306 and determine whether the measured reflectance matches an expected reflectance level. Based on the measured reflectances, the calibration system may adjust image processing (e.g., white balance), adjust an illumination source (e.g., illumination intensity, a color of illumination light, etc.), and/or take any other suitable operation.

FIG. 4A shows another illustrative configuration of fluorescence evaluation apparatus 100. FIG. 4A is similar to FIG. 1A except that in FIG. 4A fluorescence evaluation apparatus 100 further includes a plurality of distance scale markers 402 arranged along an edge of substrate 102 to form a ruler 404 for distance measurement. As shown in FIG. 4A, ruler 404 also includes distance reference numbers (e.g., “1” through “9”) indicating the distance of certain markers 402 from a left edge of substrate 102. As shown in FIG. 4A, markers 402 are arranged based on a metric scale with one millimeter intervals. However, markers 402 may be arranged based on any other scale (e.g., an imperial scale) and with any other suitable interval.

In some examples, markers 402 are printed on substrate 102 and visible under visible light. Additionally or alternatively, markers 402 comprise the fluorescence imaging agent on or in substrate 102 in a series of lines at regularly-spaced intervals. In this way, the ruler 404 may fluoresce when illuminated with fluorescence excitation illumination and thus may be viewed even when the scene is not illuminated with visible light. The concentration of the fluorescence imaging agent may be any suitable concentration, and in some examples may be similar to the concentration of the fluorescence swatch 104 having the highest concentration of the fluorescence imaging agent.

Ruler 404 may help a user (e.g., a surgeon) to measure the size of anatomical features (e.g., tumors, lymph nodes, lesions, etc.), assess the distance between two separate points at the scene (e.g., on patient tissue), and/or determine a distance from an endoscope to tissue. Further, having an object of known size in the surgical image may also help to check/confirm depth models from stereo reconstruction.

FIG. 4B shows an alternative configuration of fluorescence evaluation apparatus 100 shown in FIG. 4A. FIG. 4B is similar to FIG. 4A except that, in FIG. 4B, fluorescence evaluation apparatus 100 includes eight fluorescence swatches 104 (e.g., fluorescence swatches 104-1 through 104-8) and fluorescence swatches 104 have a block number shape ranging from “1” to “8” and each located at a position on substrate 102 corresponding to the position of a major (e.g., centimeter) marker 402. The use of number shapes for fluorescence swatches 104 conserves space on substrate 102 by eliminating the need for reference numbers on ruler 404 and may help the user remember which fluorescence swatch 104 emits fluorescence that best matches fluorescence emitted from a scene.

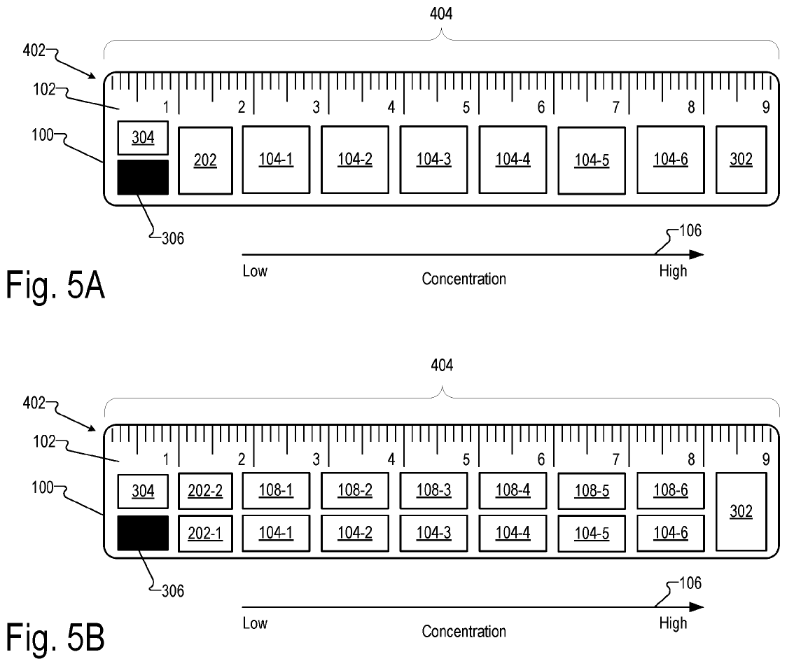

Any two or more of the features described above with reference to FIGS. 1A to 4B may be combined into a single fluorescence evaluation apparatus. For example, FIG. 5A shows another illustrative configuration of fluorescence evaluation apparatus 100 in which fluorescence swatches 104, reference fluorescence swatch 202, NIR light swatch 302, white surface swatch 304, and black surface swatch 306 are arranged on substrate 102. It will be recognized that these components may be configured and arranged on substrate 102 in any suitable way. FIG. 5B shows another illustrative configuration of fluorescence evaluation apparatus 100. FIG. 5B is similar to FIG. 5A except that in FIG. 5B fluorescence evaluation apparatus 100 further includes additional fluorescence swatches 108, a reference fluorescence swatch 202-1, and an additional reference fluorescence swatch 202-2.

In some examples of alternative configurations of fluorescence evaluation apparatus 100, fluorescence swatches 104 may have the same concentration of the fluorescence imaging agent but may be otherwise configured to emit fluorescence having different intensities. For example, fluorescence swatches 104 may have different thicknesses and/or may be arranged on substrate 102 at different depths within substrate 102, either or both of which may produce different fluorescence intensities.

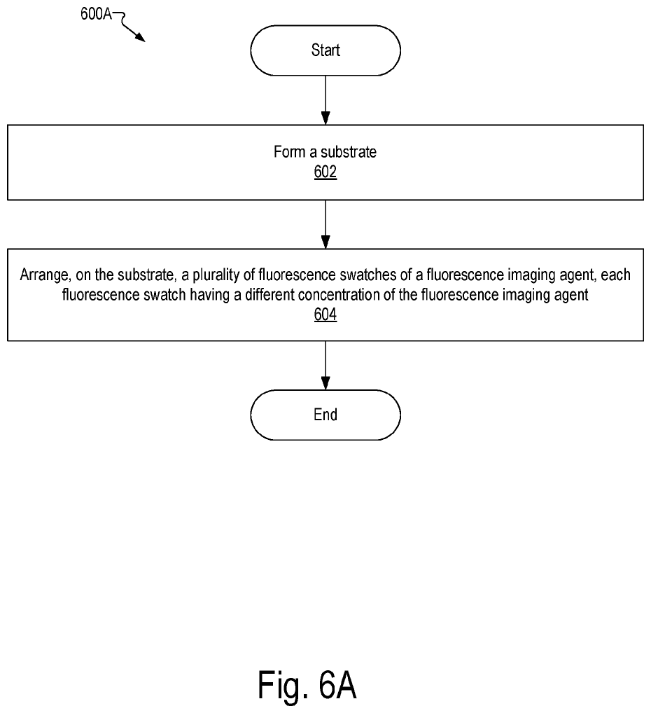

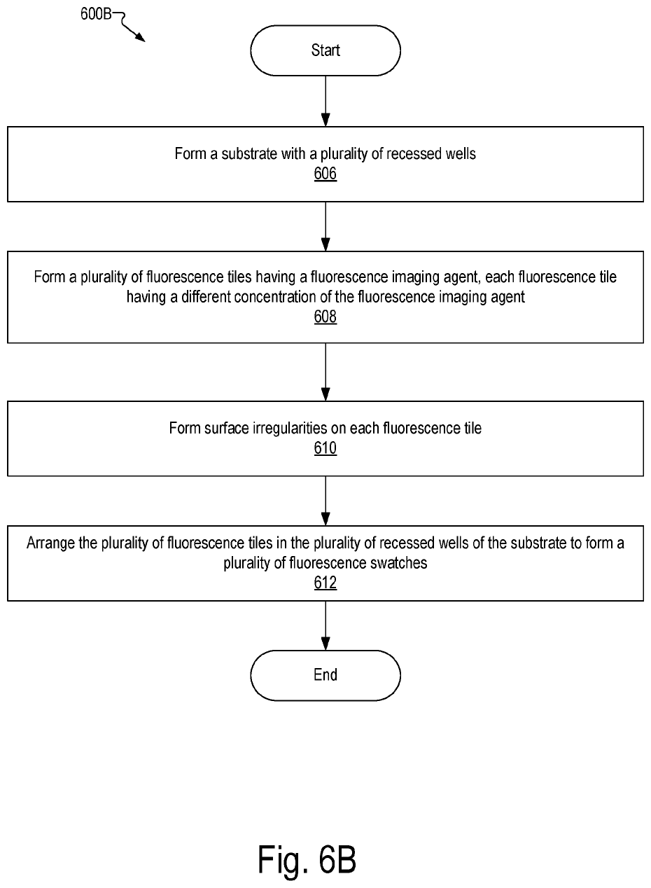

FIG. 6A shows an illustrative method 600A of making a fluorescence evaluation apparatus (e.g., fluorescence evaluation apparatus 100). While FIG. 6A illustrates steps according to one embodiment, other embodiments may omit, add to, reorder, and/or modify any of the steps shown in FIG. 6A.

At step 602, a substrate (e.g., substrate 102) is formed. Step 602 may be performed in any suitable way, such as by molding (e.g., injection molding, cast molding, compression molding, etc.), additive manufacturing (e.g., 3D printing), subtractive manufacturing, cutting, and/or any other suitable technique. In some examples, the substrate may be formed by laminating multiple layers of the same or different materials. In further examples, a surface of the substrate may be formed with a plurality of recessed wells in which a plurality of fluorescence swatches may be arranged.

At step 604, a plurality of fluorescence swatches of a fluorescence imaging agent (e.g., fluorescence swatches 104) are arranged on the substrate. Each fluorescence swatch has a different concentration of the fluorescence imaging agent. Step 604 may be performed in any suitable way, such as by embedding the fluorescence imaging agent in the substrate in different concentrations in each of a plurality of distinct regions of the substrate (e.g., by adding fluorescent mixtures of different concentrations to recessed wells previously formed in the substrate and curing the fluorescent mixtures) or by affixing separately formed fluorescence tiles in recessed wells previously formed in the substrate.

Additionally or alternatively, step 604 may comprise doping the substrate with different concentrations of the fluorescence imaging agent, coating the fluorescence imaging agent on a surface of the substrate, adhering the fluorescence imaging agent to a surface of the substrate, absorbing the fluorescence imaging agent into the substrate, and/or laminating a layer of the fluorescence imaging agent on the substrate. In some examples, the plurality of fluorescence swatches may be arranged on the substrate between different layers of a multi-layer substrate. In some examples, the fluorescence imaging agent may be encapsulated in an encapsulating agent (e.g., a surfactant, a polymer, or a dispersing agent). In further examples, method 600A may also include a step (not shown) of encapsulating the fluorescence imaging agent in an encapsulating agent.

FIG. 6B shows an illustrative method 600B. Method 600B is an implementation of method 600A that uses separately formed fluorescent tiles and that configures fluorescence swatches to quench specular reflection from the fluorescence swatches. While FIG. 6B illustrates steps according to one embodiment, other embodiments may omit, add to, reorder, and/or modify any of the steps shown in FIG. 6B.

At step 606, a substrate (e.g., substrate 102) is formed with a plurality of recessed wells in which a plurality of fluorescence tiles may be arranged. Step 606 may be performed in any suitable way, including in any way described herein (e.g., in a manner similar to step 602).

At step 608, a plurality of fluorescence tiles having a fluorescence imaging agent are formed, Each fluorescence tile has a different concentration of the fluorescence imaging agent. Step 608 may be performed in any suitable way, including any way described herein. In some examples, each fluorescence tile of the plurality of fluorescence tiles is formed by mixing a fluorescence imaging agent with a liquid base material in a desired concentration to form a fluorescence mixture and then curing the fluorescence mixture.

The base material may be any material described herein, such as a biocompatible polymer, a resin, etc. In some examples, forming the plurality of fluorescence tiles includes mixing the fluorescence imaging agent with a base material having a non-white color, Thus, when the fluorescence mixture is cured and arranged on the substrate to form a fluorescence swatch, the non-white fluorescence swatch quenches specular reflection of visible light and/or light having a wavelength shorter than a peak wavelength of fluorescence emitted by the fluorescence imaging agent.

Additionally or alternatively to using a non-white base material, forming the plurality of fluorescence tiles may include mixing, in the fluorescence mixture, an additive configured to quench specular reflection. The additive may be any additive described herein, such as an optical dust produced from an optical filter material, an absorbing agent, and/or a scattering agent.

A fluorescence mixture may be cured to form a fluorescence tile in any suitable way. In some examples, the fluorescence mixture is cured separately from the substrate (e.g., such as by 3D printing, injection molding, etc.) to form a fluorescence tile. The fluorescence tile may then be cut to size, as needed.

At step 610, surface irregularities are formed on a surface of each fluorescence tile to quench specular reflection of visible light and/or light having a wavelength shorter than a peak wavelength of fluorescence emitted by the fluorescence imaging agent. Step 610 may be performed in any suitable way, including any way described herein. In some examples, surface irregularities are formed by sanding, sand blasting, scratching, and/or etching a surface of the fluorescence tiles. Additionally or alternatively, the fluorescence tiles may be natively formed (e.g., 3D printed or injected molded) with surface irregularities.

At step 612, the plurality of fluorescence tiles are arranged in the plurality of recessed wells of the substrate. Step 612 may be performed in any suitable way. In some examples, separately formed fluorescence tiles may be attached or secured within the plurality of recessed wells by friction-fit, by an adhesive, and/or by a mechanical means (e.g., a snap-fit). In further examples, the fluorescence tiles are secured within the plurality of recessed wells by laminating a layer over the fluorescence tiles or forming a frame layer over the fluorescence tiles to hold the fluorescence tiles within the recessed wells.

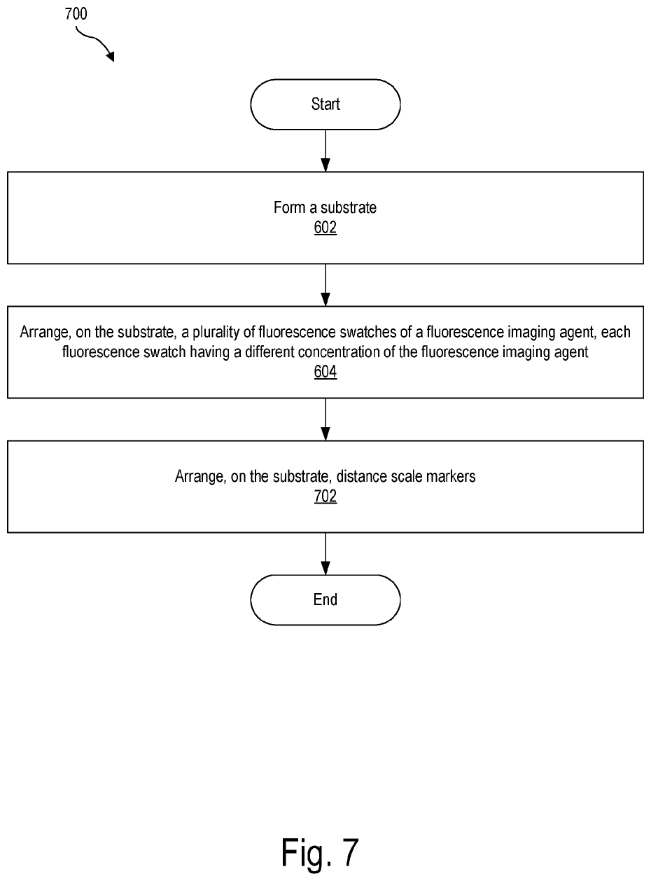

Various modifications may be made to method 600B. In some examples, step 610 may be performed after step 612 (e.g., the surface irregularities are formed in the plurality of fluorescence tiles after the fluorescence tiles are arranged in the recessed wells of the substrate). Additionally or alternatively, step 608 may be combined with step 612 by adding the fluorescence mixture to a recessed well of the substrate and curing the fluorescence mixture within the recessed well. In further modifications, step 610 may be omitted. In some examples, a layer of an optical filter material is formed on or over each fluorescence tile. For example, a layer of optical filter material may be deposited on or attached to a surface of each fluorescence tile or fluorescence swatch. Alternatively, a layer of the optical filter material may be formed over the plurality of fluorescence swatches and secured in place, such as by an adhesive or by a frame layer over the layer of optical filter material. FIG. 7 shows another illustrative method 700 of making a fluorescence evaluation apparatus. While FIG. 7 illustrates steps according to one embodiment, other embodiments may omit, add to, reorder, and/or modify any of the steps shown in FIG. 7. Method 700 is similar to method 600A except that method 700 includes an additional step 702.

At step 702, distance scale markers (e.g., markers 402) are arranged on the substrate. Step 702 may be performed in any way described herein, such as printing, etching, coloring, or marking. In some examples in which the distance scale markers comprise a fluorescence imaging agent, the distance scale markers may be arranged on the substrate in any similar to arranging the fluorescence swatches on the substrate. For example, the fluorescence imaging agent may be embedded in or on the substrate at positions corresponding to the distance scale markers (e.g., in a series of lines at regularly-spaced intervals).

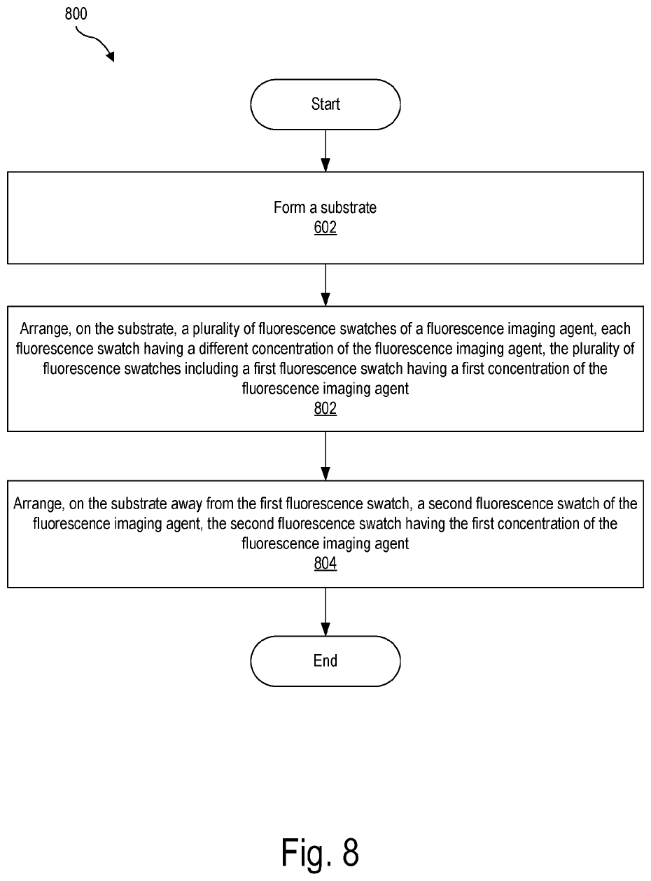

FIG. 8 shows another illustrative method 800 of making a fluorescence evaluation apparatus (e.g., fluorescence evaluation apparatus 100). While FIG. 8 illustrates steps according to one embodiment, other embodiments may omit, add to, reorder, and/or modify any of the steps shown in FIG. 8. Method 800 is similar to method 600A except that in method 800 step 604 is replaced with step 802 and method 800 includes an additional step 804.

At step 802, a plurality of fluorescence swatches of a fluorescence imaging agent (e.g., fluorescence swatches 104) are arranged on the substrate. Each fluorescence swatch has a different concentration of the fluorescence imaging agent. The plurality of fluorescence swatches includes a first fluorescence swatch (e.g., fluorescence swatch 104-6) having a first concentration of the fluorescence imaging agent.

At step 804, a second fluorescence swatch of the fluorescence imaging agent (e.g., reference fluorescence swatch 202) is arranged on the substrate away from the first fluorescence swatch. The second fluorescence swatch has the first concentration of the fluorescence imaging agent. The second fluorescence swatch may be arranged on the substrate in any way described above with regard to the plurality of fluorescence swatches.

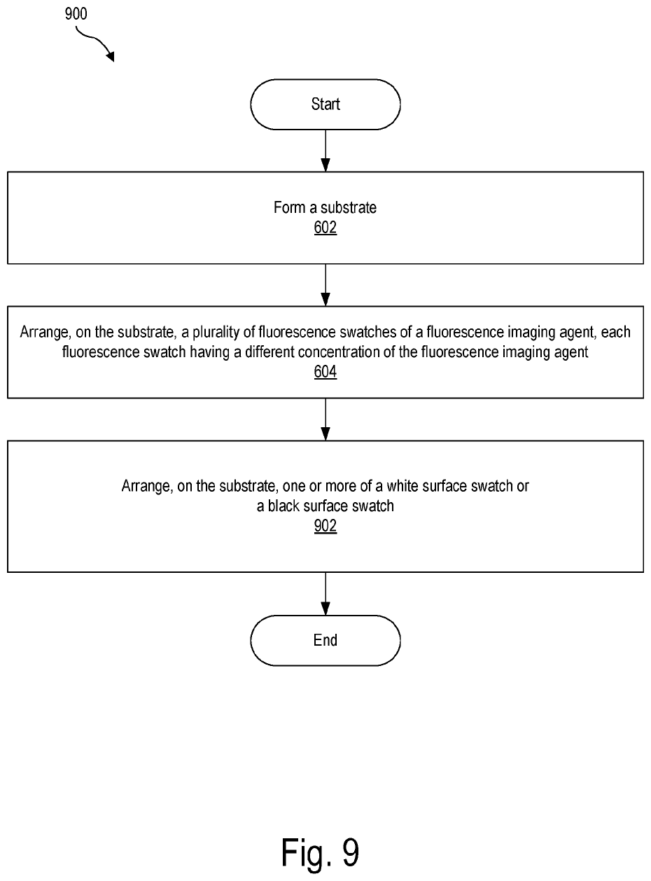

FIG. 9 shows another illustrative method 900 of making a fluorescence evaluation apparatus (e.g., fluorescence evaluation apparatus 100). While FIG. 9 illustrates steps according to one embodiment, other embodiments may omit, add to, reorder, and/or modify any of the steps shown in FIG. 9. Method 900 is similar to method 600A except that method 900 includes an additional step 902.

At step 902, a white surface swatch (e.g., white surface swatch 304) and/or a black surface swatch (e.g., black surface swatch 306) is/are arranged on the substrate. Step 902 may be performed in any suitable way, such as by embedding, doping, coating, painting, printing, layering, or adhering the white surface swatch and/or the black surface swatch in or on the substrate.

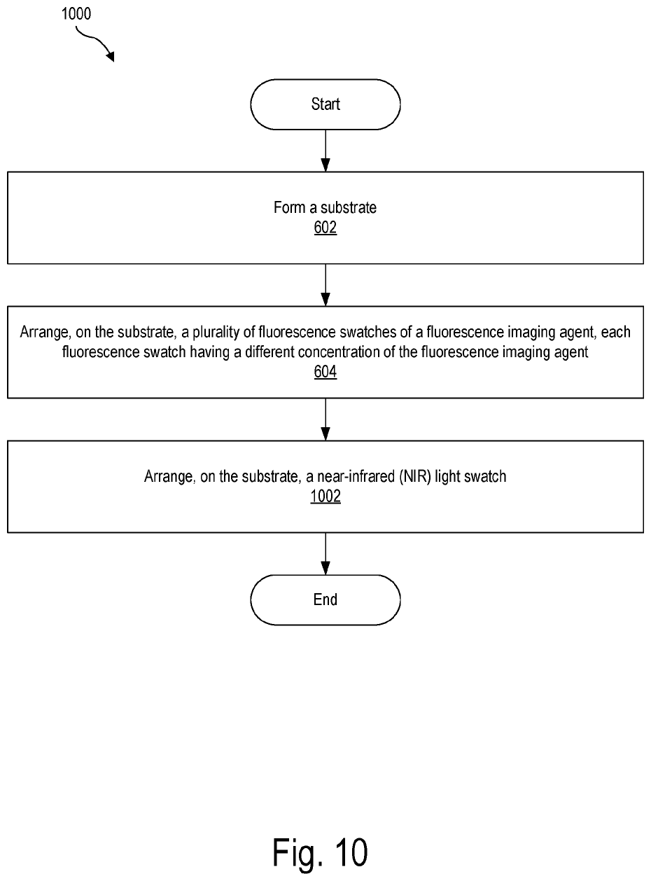

FIG. 10 shows another illustrative method 1000 of making a fluorescence evaluation apparatus (e.g., fluorescence evaluation apparatus 100). While FIG. 10 illustrates steps according to one embodiment, other embodiments may omit, add to, reorder, and/or modify any of the steps shown in FIG. 10. Method 1000 is similar to method 600A except that method 1000 includes an additional step 1002.

At step 1002, a NIR light swatch (e.g., NIR light swatch 302) is arranged on the substrate. The NIR light swatch includes an up-converting fluorescence imaging agent configured to emit visible light when excited by NIR light. Step 1002 may be performed in any suitable way, such as by embedding, doping, coating, painting, printing, layering, or adhering the NIR light swatch in or on the substrate.

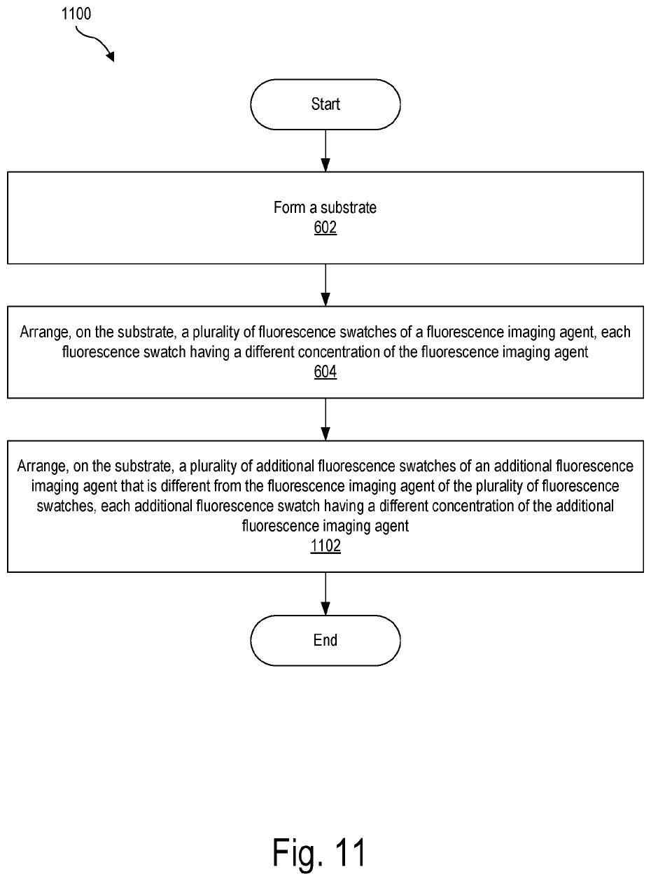

FIG. 11 shows another illustrative method 1100 of making a fluorescence evaluation apparatus (e.g., fluorescence evaluation apparatus 100). While FIG. 11 illustrates steps according to one embodiment, other embodiments may omit, add to, reorder, and/or modify any of the steps shown in FIG. 11. Method 1100 is similar to method 600A except that method 1100 includes an additional step 1102.

At step 1102, a plurality of additional fluorescence swatches of an additional fluorescence imaging agent (e.g., fluorescence swatches 108) are arranged on the substrate. The additional fluorescence imaging agent is different from the fluorescence imaging agent, and each additional fluorescence swatch has a different concentration of the additional fluorescence imaging agent. The plurality of additional fluorescence swatches may be arranged on the substrate in any way described above with regard to the plurality of fluorescence swatches.

It will be recognized that any steps described above with regard to methods 600A to 1100 may be combined to make any of the fluorescence evaluation apparatuses described herein, including fluorescence evaluation apparatus 100 shown in FIGS. 5A and 5B.

Fluorescence evaluation apparatuses described herein (e.g., fluorescence evaluation apparatus 100) may be used to evaluate fluorescence that is emitted from a scene and captured by an imaging device. For example, fluorescence emitted from a scene may be evaluated during a medical procedure to assess or estimate the in situ concentration of a fluorescence imaging agent (e.g., ICG) present in patient tissue and/or to assess perfusion of the tissue. Fluorescence emitted from a scene may also be evaluated to assess operation of the imaging device (e.g., sensitivity of the imaging device to fluorescence), determine the efficiency of fluorescence detection by the imaging device, and/or calibrate the imaging device. To facilitate understanding of various possible uses of a fluorescence evaluation apparatus, an illustrative imaging system and imaging device will now be described.

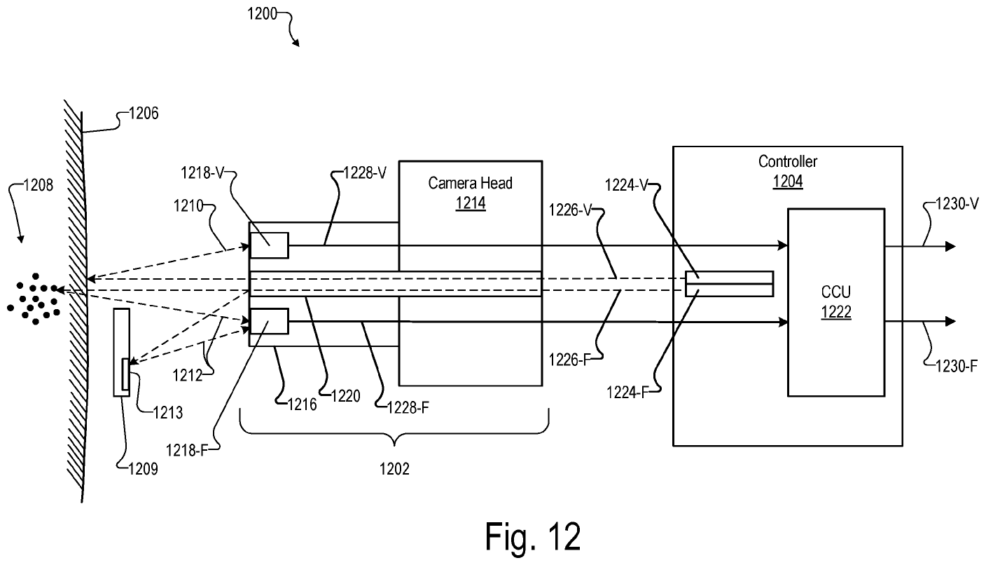

FIG. 12 shows a functional diagram of an illustrative imaging system 1200 that may be used in accordance with the apparatuses, systems, and methods described herein to capture visible light images of a scene and fluorescence images of the scene. As shown, imaging system 1200 includes an imaging device 1202 and a controller 1204. Imaging system 1200 may include additional or alternative components as may serve a particular implementation. For example, imaging system 1200 may include various optical and/or electrical signal transmission components (e.g., wires, lenses, optical fibers, choke circuits, waveguides, etc.), a cable that houses electrical wires and/or optical fibers and that is configured to interconnect imaging device 1202 and controller 1204, etc. While imaging system 1200 shown and described herein comprises a fluorescence imaging system integrated with a visible light imaging system, imaging system 1200 may alternatively be implemented as a standalone fluorescence imaging system configured to capture only fluorescence images of the scene. Accordingly, components of imaging system 1200 that function only to capture visible light (e.g., white light) images may be omitted. In some examples a standalone fluorescence imaging system may be physically integrated with a visible light imaging system, such as by inserting the fluorescence imaging system into an assistance port of an endoscope.

As shown in FIG. 12, imaging device 1202 may be used to capture visible light images and fluorescence images of a scene. In some examples, the scene may include an area associated with a body on or within which a fluorescence-guided medical procedure is being performed (e.g., a body of a live human or animal, a human or animal cadaver, a portion of human or animal anatomy, tissue removed from human or animal anatomies, non-tissue work pieces, training models, etc.). In other examples, the scene may be a non-medical scene, such as a scene captured for calibration or operational assessment purposes. As shown in FIG. 12, the scene includes patient tissue 1206 and a fluorescence imaging agent 1208 present within tissue 1206. The scene may also include other objects not shown in FIG. 12, such as a fluorescence evaluation apparatus 1209 (e.g., fluorescence evaluation apparatus 100). Imaging device 1202 may capture visible light images of tissue 1206 and/or other objects within the scene based on visible light 1210 reflected by tissue 1206 and other objects at the scene, and may capture fluorescence images based on fluorescence 1212 emitted by fluorescence imaging agent 1208 and by one or more fluorescence swatches 1213 of fluorescence evaluation apparatus 1209.

Imaging device 1202 may be implemented by any suitable device configured to capture images of a scene. In some examples, imaging device 1202 is implemented by an endoscope. Imaging device 1202 includes a camera head 1214, a shaft 1216 coupled to and extending away from camera head 1214, image sensors 1218 (e.g., a visible light sensor 1218-V and a fluorescence detection sensor 1218-F), and an illumination channel 1220. Imaging device 1202 may be manually handled and controlled (e.g., by a surgeon performing a surgical procedure on a patient). Alternatively, camera head 1214 may be coupled to a manipulator arm of a computer-assisted surgical system and controlled using robotic and/or teleoperation technology. The distal end of shaft 1216 may be positioned at or near the scene that is to be imaged by imaging device 1202. For example, the distal end of shaft 1216 may be inserted into a patient via a cannula.

Visible light sensor 1218-V is configured to detect (e.g., capture, collect, sense, or otherwise acquire) visible light 1210 reflected from tissue 1206 and any objects included within the scene, such as surgical instruments. As will be explained below, visible light sensor 1218-V may convert the detected visible light into data representative of one or more visible light images. Visible light sensor 1218-V may be implemented by any suitable image sensor, such as a charge coupled device (CCD) image sensor, a complementary metal-oxide semiconductor (CMOS) image sensor, or the like. In some examples, as shown in FIG. 12, visible light sensor 1218-V is positioned at the distal end of shaft 1216. Alternatively, visible light sensor 1218-V may be positioned closer to a proximal end of shaft 1216, inside camera head 1214, or outside imaging device 1202 (e.g., inside controller 1204). In these alternative configurations, optics (e.g., lenses, optical fibers, etc.) included in shaft 1216 and/or camera head 1214 may convey light from the scene to visible light sensor 1218-V.

Fluorescence detection sensor 1218-F is configured to detect (e.g., capture, collect, sense, or otherwise acquire) fluorescence 1212 emitted by fluorescence imaging agent 1208. Fluorescence 1212 may have a wavelength in an ultraviolet, visible, and/or infrared region. As will be explained below, fluorescence detection sensor 1218-F may convert the detected fluorescence 1212 into data representative of one or more fluorescence images. Fluorescence detection sensor 1218-F may be implemented by any suitable sensor configured to detect fluorescence 1212. Suitable sensors may include, without limitation, CCD image sensors, CMOS image sensors, photodetectors based on time-correlated single photon counting (TCSPC) (e.g., a single photon counting detector, a photo multiplier tube (PMT), a single photon avalanche diode (SPALL) detector, etc.), photodetectors based on time-gating (e.g., intensified CCDs), time-of-flight sensors, streak cameras, and the like.

Fluorescence detection sensor 1218-F may be positioned at the distal end of shaft 1216, or it may alternatively be positioned closer to the proximal end of shaft 1216, inside camera head 1214, or outside imaging device 1202 (e.g., inside controller 1204). In these alternative configurations, optics included in shaft 1216 and/or camera head 1214 may convey fluorescence 1212 from the scene to fluorescence detection sensor 1218-F. In some examples, fluorescence detection sensor 1218-F may share optics with visible light sensor 1218-V.

Fluorescence detection sensor 1218-F may capture images of all or part of the scene captured by visible light sensor 1218-V. In some examples, the field of view of fluorescence detection sensor 1218-F may be the same as visible light sensor 1218-V but may differ slightly (due to its position within shaft 1216) without loss of utility.

In some examples, imaging device 1202 is stereoscopic, in which case visible light sensor 1218-V includes two sensors configured to capture left and right visible images of the scene. Likewise, fluorescence detection sensor 1218-F may include two distinct sensors configured to capture left and right fluorescence images of the scene. In additional or alternative examples, sensors 1218 are each configured to capture both visible light images and fluorescence images (e.g., a left sensor 1218 is configured to capture both left visible light images and left fluorescence images, and a right sensor 1218 is configured to capture both right visible light images and right fluorescence images). In yet other examples, imaging device 1202 is monoscopic, in which case visible light sensor 1218-V and/or fluorescence detection sensor 1218-F are configured to capture a single visible light image and a single fluorescence image, respectively.

Illumination channel 1220 may be implemented by one or more optical components (e.g., optical fibers, light guides, lenses, etc.). Illumination (e.g., visible light and/or fluorescence excitation illumination (e.g., NIR light)) may be provided to the scene by way of illumination channel 1220 to illuminate the scene.

Controller 1204 may be implemented by any suitable combination of hardware and software configured to control and/or interface with imaging device 1202. For example, controller 1204 may be at least partially implemented by a computing device included in a computer-assisted surgical system. Controller 1204 includes a camera control unit (CCU) 1222 and illumination sources 1224 (e.g., a visible light illumination source 1224-V and a fluorescence excitation illumination source 1224-F). Controller 1204 may include additional or alternative components as may serve a particular implementation. For example, controller 1204 may include circuitry configured to provide power to components included in imaging device 1202. In some examples, CCU 1222 and/or illumination sources 1224 are alternatively included in imaging device 1202 (e.g., in camera head 1214).

CCU 1222 may be configured to control (e.g., define, adjust, configure, set, etc.) operation of image sensors 1218. CCU 1222 may also be configured to receive and process image data from image sensors 1218. While CCU 1222 is shown in FIG. 12 to be a single unit, CCU 1222 may alternatively be implemented by multiple CCUs each configured to control distinct image streams (e.g., a visible light image stream, a fluorescence image stream, a right-side fluorescence image stream, a left-side image fluorescence stream, etc.).

Illumination sources 1224 may be configured to generate and emit illumination 1226. Illumination 1226 (which may also be referred herein to as light) may travel by way of illumination channel 1220 to a distal end of shaft 1216, where illumination 1226 exits to illuminate the scene. Illumination 1226 generated by visible light illumination source 1224-V may include visible light 1226-V having one or more color components or a continuous spectrum of light (e.g., white light). Illumination generated by fluorescence excitation illumination source 1224-F may include fluorescence excitation illumination 1226-F configured to excite fluorescence imaging agent 1208. Fluorescence excitation illumination 1226-F may include one or more broadband spectra of light or may include one or more discrete wavelengths of light.

Illumination sources 1224 may be configured to operate in accordance with one or more definable (e.g., adjustable) parameters (e.g., parameters that specify a wavelength or wavelength band, a waveform, an intensity, a frequency, a pulse-width, a period, a modulation, etc.), Illumination sources 1224 may be implemented by any suitable device, such as a flash lamp, laser source, laser diode, light-emitting diode, and the like. While each illumination source 1224 is shown to be a single device in controller 1204, each illumination source 1224 may alternatively include multiple illumination sources each configured to generate and emit differently configured illumination. Alternatively, while illumination sources 1224 are shown in FIG. 12 to be multiple units, illumination sources 1224 may instead be implemented by a single unit configured to emit both visible light 1226-V and fluorescence excitation illumination 1226-F.

To capture one or more images of a scene, controller 1204 (or any other suitable computing device) may activate illumination sources 1224 and image sensors 1218. While activated, illumination sources 1224 concurrently emit illumination 1226, which travels via illumination channel 1220 to the scene. Visible light sensor 1218-V detects visible light 1210 (e.g., the portion of visible light 1226-V that is reflected from one or more surfaces in the scene, such as tissue 1206), and fluorescence detection sensor 1218-F detects fluorescence 1212 that is emitted by fluorescence imaging agent 1208 upon excitation by fluorescence excitation illumination 1226-F.

Visible light sensor 1218-V (and/or other circuitry included in imaging device 1202) may convert the detected visible light 1210 into visible light image data 1228-V representative of one or more visible light images of the scene. Similarly, fluorescence detection sensor 1218-F (and/or other circuitry included in imaging device 1202) may convert the detected fluorescence 1212 into fluorescence image data 1228-F representative of one or more fluorescence images of the scene. Image data 1228 (e.g., visible light image data 1228-V and fluorescence image data 1228-F) may have any suitable format.

Image data 1228 is transmitted from image sensors 1218 to CCU 1222. Image data 1228 may be transmitted by way of any suitable communication link between image sensors 1218 and CCU 1222. For example, image data 1228 may be transmitted by way of wires included in a cable that interconnects imaging device 1202 and controller 1204. Additionally or alternatively, image data 1228 may be transmitted by way of one or more optical fibers. CCU 1222 may process (e.g., packetize and/or format) image data 1228 and output processed image data 1230 (e.g., processed visible light image data 1230-V corresponding to visible light image data 1228-V and processed fluorescence image data 1230-F corresponding to fluorescence image data 1228-F). CCU 1222 may transmit processed image data 1230 to an image processor (not shown) for further processing.

The image processor may be implemented by one or more computing devices external to imaging system 1200, such as one or more computing devices included in a computer-assisted surgical system. Alternatively, the image processor may be included in controller 1204. The image processor may prepare processed image data 1230 for display by one or more display devices (e.g., in the form of one or more still images and/or video content). For example, the image processor may generate, based on processed visible light image data 1230-V, a plurality of visible light images, which may be sequentially output to form a visible light image stream. The visible light images may include full color images and/or grayscale images. The image processor may also generate, based on processed fluorescence image data 1230-F, a plurality of fluorescence images, which may be sequentially output to form a fluorescence image stream. System 1200 may direct one or more display devices to then display the visible light image stream and/or the fluorescence image stream.

In some examples, the image processor may combine (e.g., blend) processed visible light image data 1230-V and processed fluorescence image data 1230-F to generate a plurality of augmented images, which may be sequentially output to form an augmented image stream for display by one or more display devices. An augmented image may display fluorescing regions (derived from processed fluorescence image data 1230-F) artificially colored, such as green or blue, to highlight the fluorescing regions. Additionally, the image processor may be configured to selectively apply a gain to a fluorescence image to adjust (e.g., increase or decrease) the illumination intensity of the fluorescing regions. Imaging system 1200 may direct one or more display devices to display the augmented image stream.

In some examples, the image processor may operate in accordance with one or more definable (e.g., adjustable) parameters. For example, the image processor may be configured to set a color of fluorescing regions, perform white balance, correct processed image data 1230, and perform other similar operations.

As mentioned, a fluorescence evaluation apparatus (e.g., fluorescence evaluation apparatus 1209) may be used during a medical procedure for evaluation of fluorescence (e.g., fluorescence 1212) emitted by a fluorescence imaging agent (e.g., fluorescence imaging agent 1208) present in a scene and captured by an imaging device (e.g., imaging device 1202). In some scenarios, it may be desirable for a user (e.g., a surgeon or an anesthesiologist) to know the concentration of the fluorescence imaging agent in patient tissue. As will now be explained, detected fluorescence may be evaluated with the aid of a fluorescence evaluation apparatus to assess the in situ concentration of a fluorescence imaging agent (e.g., ICG) present in patient tissue.

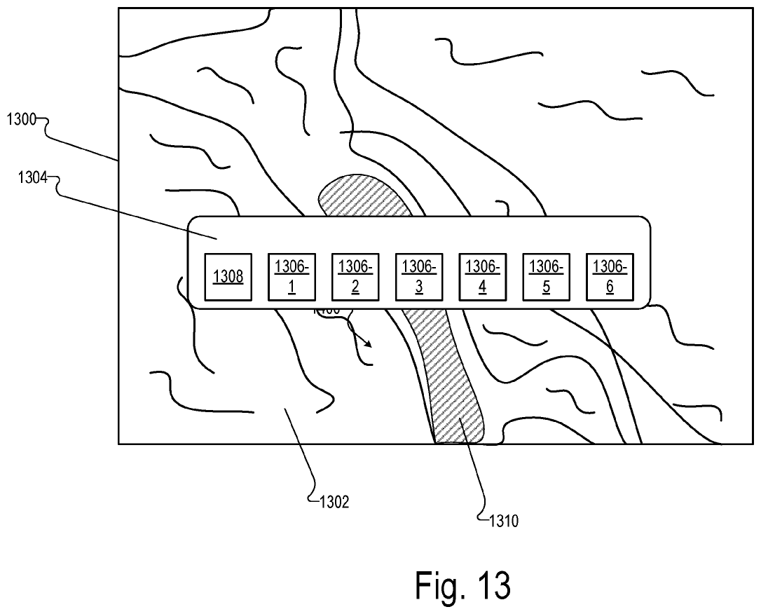

FIG. 13 shows an illustrative augmented image 1300 captured by an imaging device (e.g., imaging device 1202) in which a visible light image (e.g., a black-and-white image) is augmented with a fluorescence image. Augmented image 1300 depicts a scene comprising tissue 1302 and a fluorescence evaluation apparatus 1304 when the scene is illuminated with visible light and fluorescence excitation illumination. While the assessment of in situ fluorescence imaging agent concentration is described with reference to augmented image 1300, the assessment may alternatively be performed with a fluorescence image (e.g., an image generated based only on detected fluorescence and not on visible light).

Fluorescence evaluation apparatus 1304 is shown resting on tissue 1302, but fluorescence evaluation apparatus 1304 may alternatively be held by a user or a surgical instrument (e.g., a surgical instrument connected to a manipulator arm of a computer-assisted surgical system). As shown, fluorescence evaluation apparatus 1304 includes a plurality of fluorescence swatches 1306 (e.g., fluorescence swatches 1306-1 through 1306-6) of a fluorescence imaging agent and a reference fluorescence swatch 1308 of the fluorescence imaging agent. However, fluorescence evaluation apparatus 1304 may be implemented by any other fluorescence evaluation apparatus described herein. Fluorescence swatches 1306 and reference fluorescence swatch 1308 emit fluorescence based on the fluorescence excitation illumination. An intensity of the fluorescence emitted by each fluorescence swatch 1306 and by reference fluorescence swatch 1308 varies based on the concentration of the fluorescence imaging agent in each fluorescence swatch 1306 and in reference fluorescence swatch 1308. Fluorescence swatches 1306 are arranged, from fluorescence swatch 1306-1 to fluorescence swatch 1306-6, in order of increasing concentration. Reference fluorescence swatch 1308 may have the same concentration of the fluorescence imaging agent as fluorescence swatch 1306-6.

Augmented image 1300 also shows a target region 1310 for evaluation (indicated by right-left cross-hatching). Target region 1310 is a region of tissue 1302 that includes a fluorescence imaging agent that emits fluorescence based on the fluorescence excitation illumination. An intensity of the fluorescence emitted from target region 1310 is based on the concentration of the fluorescence imaging agent in target region 1310. The fluorescence emitted by fluorescence swatches 1306, reference fluorescence swatch 1308, and target region 1310 is detected by the imaging device and may be pseudo-colored in augmented image 1300 to highlight the fluorescing regions.

Before assessing the in situ concentration of the fluorescence imaging agent in target region 1310, the user may confirm the proper positioning of fluorescence evaluation apparatus 1304 by comparing the intensity of fluorescence emitted from fluorescence swatch 1306-6 with the intensity of fluorescence emitted from reference fluorescence swatch 1308. If the intensities are substantially the same, the user may assume that fluorescence evaluation apparatus 1304 is properly positioned and that fluorescence emitted from fluorescence swatches 1306 is not influenced by variations in the distance between fluorescence swatches 1306 and the fluorescence excitation illumination source (e.g., a distal end of the imaging device). If, however, the intensities are different, the user may easily determine that adjustment of fluorescence evaluation apparatus 1304 should be made and make the appropriate adjustments.

To assess the in situ concentration of the fluorescence imaging agent in target region 1310, the user may compare the intensity of fluorescence from target region 1310 with the intensity of fluorescence from fluorescence swatches 1306. Based on the comparison, the user may identify a particular fluorescence swatch 1306 having a fluorescence intensity that corresponds (e.g., is substantially the same as) with the fluorescence intensity from target region 1310. For example, the user may determine, based on augmented image 1300, that fluorescence emitted from target region 1310 corresponds more closely with fluorescence emitted from fluorescence swatch 1306-5. Accordingly, the user may determine that a concentration of the fluorescence imaging agent in target region 1310 is relatively high.

In the examples above, fluorescence evaluation apparatus 1304 provides a relative estimation of the in situ concentration of the fluorescence imaging agent. In other examples, the concentration of the fluorescence imaging agent in fluorescence swatch 1306-5 may be known (e.g., printed on fluorescence evaluation apparatus 1304, supplied with an instruction sheet for fluorescence evaluation apparatus 1304, stored in a memory of a computing device and accessible to the user, etc.). Accordingly, the user may be able to estimate a value of the in situ concentration of the fluorescence imaging agent in target region 1310 based on the known concentration of the fluorescence imaging agent in fluorescence swatch 1306-5.

In some scenarios, it may also be desirable for a user (e.g., a surgeon) to assess perfusion (e.g., blood flow) in a region of tissue in the scene. For example, colon cancer may be treated by removing a tumor and then performing a resection of the intestines with an intestinal anastomosis. However, if the anastomosis does not heal properly, the anastomosis may leak and the patient may develop sepsis. Assessing tissue perfusion in tissue during a surgical procedure may help determine whether the anastomosis is successful and the tissue is likely to heal.

As will now be described, fluorescence emitted from tissue may be evaluated with the aid of a fluorescence evaluation apparatus to assess perfusion. The fluorescence evaluation apparatus allows a user to quickly and accurately determine whether a target region of tissue has healthy or poor perfusion by comparing fluorescence from the target region with fluorescence from a reference region having healthy perfusion.

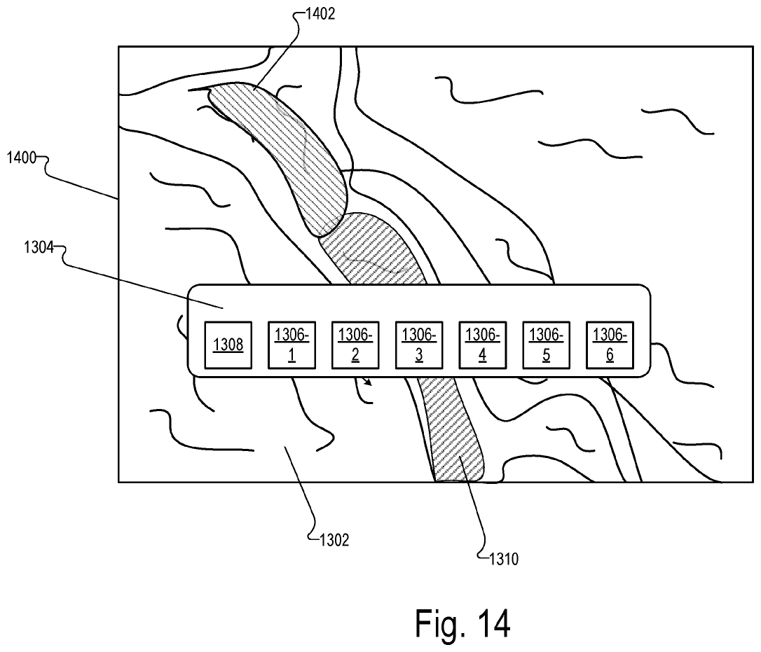

FIG. 14 shows an illustrative augmented image 1400 that may be used for in vivo assessment of tissue perfusion. While the assessment of in vivo tissue perfusion is described with reference to augmented image 1400, the assessment may alternatively be performed with a fluorescence image. FIG. 14 is similar to FIG. 13 except that in FIG. 14 augmented image 1400 also shows a reference region 1402 (indicated by left-right cross-hatching) adjacent to target region 1310. Reference region 1402 is another region of tissue 1302 and may be identified and selected by the user as a region of tissue 1302 having healthy perfusion. For example, reference region 1402 may be a portion of a colon located upstream (in the direction of arterial blood flow) from target region 1310. Target region 1310 may be another portion of the colon located downstream (in the direction of arterial blood flow) from reference region 1402. In alternative examples, reference region 1402 and target region 1310 are not adjacent to or connected with one another.

Reference region 1402 also includes the fluorescence imaging agent that emits fluorescence based on the fluorescence excitation illumination. An intensity of the fluorescence emitted from reference region 1402 is based on the concentration of the fluorescence imaging agent in reference region 1402. The fluorescence emitted by reference region 1402 is detected by the imaging device and may be pseudo-colored in augmented image 1400 to highlight the fluorescing region.

Before assessing the in vivo perfusion of target region 1310, the position of fluorescence evaluation apparatus 1304 may also be confirmed and adjusted, as appropriate, as described above.

To assess the in vivo perfusion of target region 1310, the user may compare the intensity of fluorescence from target region 1310 with the intensity of fluorescence from fluorescence swatches 1306. Based on the comparison, the user may identify a particular fluorescence swatch 1306 having a fluorescence intensity that corresponds to the fluorescence intensity from target region 1310. The user may also compare the intensity of fluorescence from reference region 1402 with the intensity of fluorescence from fluorescence swatches 1306. Based on the comparison, the user may identify another particular fluorescence swatch 1306 having a fluorescence intensity that corresponds to the fluorescence intensity from reference region 1402. The user may then assess the perfusion of target region 1310 by comparing the identified fluorescence swatch 1306 corresponding to target region 1310 with the identified fluorescence swatch 1306 corresponding to reference region 1402.

To illustrate, the user may determine, based on augmented image 1400, that fluorescence emitted from target region 1310 corresponds more closely with fluorescence emitted from fluorescence swatch 1306-5. If the user determines, based on augmented image 1400, that fluorescence emitted from reference region 1402 corresponds more closely with fluorescence emitted from fluorescence swatch 1306-4, 1306-5, or 1306-6, the user may determine that target region 1310 has healthy perfusion. On the other hand, if the user determines, based on augmented image 1400, that fluorescence emitted from reference region 1402 corresponds more closely with fluorescence emitted from fluorescence swatch 1306-1, 1306-2, or 1306-3, the user may determine that target region 1310 has poor perfusion.

In the examples described above, fluorescence evaluation apparatus 1304 serves as an objective reference standard for evaluating fluorescence emitted from a scene. Using the fluorescence evaluation apparatus 1304 as a reference standard enables the user to assess quickly and easily the in situ concentration of the fluorescence imaging agent and/or assess in vivo tissue perfusion.

In some examples, fluorescence evaluation apparatus 1304 may be used in conjunction with a computing system configured to automatically evaluate fluorescence emitted from a scene. For example, the in situ assessment of the fluorescence imaging agent concentration and/or the in vivo assessment of tissue perfusion may be performed automatically by a fluorescence evaluation system.

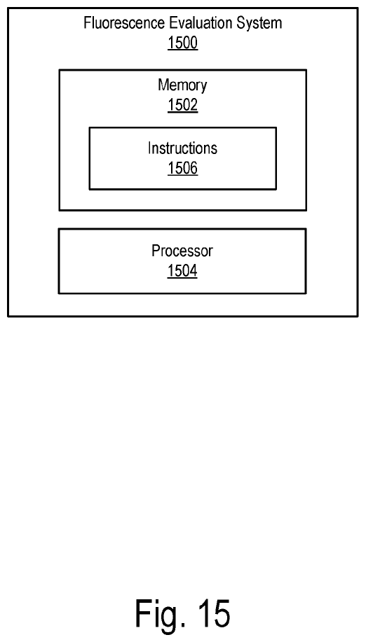

FIG. 15 shows an illustrative fluorescence evaluation system 1500 (system 1500) that may be configured to evaluate, with the aid of a fluorescence evaluation apparatus, fluorescence emitted from a scene. System 1500 may be included in, implemented by, or connected to any surgical systems or other computing systems described herein. For example, system 1500 may be implemented by a computer-assisted surgical system. As another example, system 1500 may be implemented by a stand-alone computing system communicatively coupled to a computer-assisted surgical system.

As shown, system 1500 includes, without limitation, a memory 1502 and a processor 1504 selectively and communicatively coupled to one another. Memory 1502 and processor 1504 may each include or be implemented by hardware and/or software components (e.g., processors, memories, communication interfaces, instructions stored in memory for execution by the processors, etc.). For example, memory 1502 and processor 1504 may be implemented by any component in a computer-assisted surgical system. In some examples, memory 1502 and processor 1504 may be distributed between multiple devices and/or multiple locations as may serve a particular implementation.

Memory 1502 may maintain (e.g., store) executable data used by processor 1504 to perform any of the operations described herein. For example, memory 1502 may store instructions 1506 that may be executed by processor 1504 to perform any of the operations described herein. Instructions 1506 may be implemented by any suitable application, software, code, and/or other executable data instance. Memory 1502 may also maintain any data received, generated, managed, used, and/or transmitted by processor 1504.

Processor 1504 may be configured to perform (e.g., execute instructions 1506 stored in memory 1502 to perform) various operations associated with evaluating fluorescence from a scene, such as assessing in situ concentration of a fluorescence imaging agent, assessing in vivo tissue perfusion, assessing operation of a fluorescence imaging mode of an imaging device, and calibrating an imaging device. Operations that may be performed by processor 1504 are described herein. In the description that follows, any references to operations performed by system 1500 may be understood to be performed by processor 1504 of system 1500.

In some examples, system 1500 may be configured to automatically identify a particular fluorescence swatch (e.g., a fluorescence swatch 1306 that corresponds to a particular region of interest (e.g., target region 1310 or reference region 1402). To this end, system 1500 may obtain image data (e.g., image data 1228 or image data 1230) representative of an image (e.g., augmented image 1300 or 1400) of a scene including fluorescence evaluation apparatus 1304. The image data may include fluorescence image data (e.g., fluorescence image data 1228-F or processed fluorescence image data 1230-F) representative of a fluorescence image or augmented image data (e.g., a combination of visible light image data and fluorescence image data) representative of an augmented image.

System 1500 may identify, based on the image data, the region of interest. System 1500 may identify the region of interest in any suitable way, such as by segmenting the image data. Any suitable image segmentation technique may be performed. In some examples, system 1500 may identify the region of interest automatically based on the image segmentation and based on surgical procedure data indicating a type of surgical procedure performed and/or a particular anatomical feature of interest for the surgical procedure. Additionally or alternatively, system 1500 may identify the region of interest based on user input indicating the region of interest. For example, referring to FIG. 13, a user may place a fiducial marker on target region 1310 in augmented image 1300 or draw a boundary around target region 1310. Any other suitable input may be used to identify the region of interest. Based on the user input and the image segmentation, system 1500 may identify the particular region of interest.

System 1500 may also identify, in the image, each fluorescence swatch of the fluorescence evaluation apparatus. System 1500 may identify each fluorescence swatch in any suitable way. In some examples, system 1500 may identify each fluorescence swatch based on the segmentation of the image. In some embodiments, the fluorescence swatches may each have a unique pattern or shape configured for detection and identification during image segmentation. In additional or alternative examples, system 1500 may identify each fluorescence swatch based on user input, in a manner similar to identification of the region of interest based on user input.

System 1500 may determine an intensity level of the detected fluorescence from the region of interest and from each of the fluorescence swatches. System 1500 may determine the intensity of detected fluorescence in any suitable way. For example, system 1500 may determine the intensity based on a detection result of the imaging device (e.g. imaging device 1202) and/or based on fluorescence image data (e.g., fluorescence image data 1228-F or processed fluorescence image data 1230-F) generated by the imaging device. System 1500 may then compare the fluorescence intensity level of the region of interest and of each fluorescence swatch to identify a particular fluorescence swatch that corresponds to the region of interest.

System 1500 may indicate the particular fluorescence swatch that corresponds to the region of interest. For example, system 1500 may update the image to change the pseudo-coloring of the particular fluorescence swatch (and, optionally, the region of interest) to be different from the other fluorescence swatches, display a marker or indicator on the particular fluorescence swatch, and/or perform any other suitable notification operation (e.g., provide a visual or textual message, etc.).

The automatic detection of a fluorescence swatch that corresponds to the region of interest may be used for assessment of the in situ concentration of a fluorescence imaging agent in a target region. For example, system 1500 may determine, based on the particular fluorescence swatch identified as corresponding to the target region, a fluorescence imaging agent concentration associated with the particular fluorescence swatch. As an example, system 1500 may access (e.g., from local memory or from a remote computing system), concentration data representative of a concentration associated with each fluorescence swatch 1306 of fluorescence evaluation apparatus 1304. System 1500 may then provide the concentration data for display, such as in augmented image 1300 (or some other display device or display window).

In the example just described, the intensity level of the detected fluorescence is presumed to be based only on the in situ concentration of the fluorescence imaging agent. In practice, however, the intensity level of the detected fluorescence may also depend on various other parameters, such as patient age, gender, weight, disease state, and cardiac condition (e.g., heart rate), as well as distance from the endoscope to the tissue and to the fluorescence evaluation apparatus. Accordingly, system 1500 may be configured to determine the in situ concentration of the fluorescence imaging agent based on one or more additional parameters. For example, system 1500 may obtain (e.g., by user input and/or from local memory or a remote computing system) patient data and/or surgical session data (e.g., data indicating endoscope position relative to tissue and/or the fluorescence evaluation apparatus) and apply the data to a model that models in situ concentration of a fluorescence imaging agent based on various parameters.

The automatic detection of a fluorescence swatch that corresponds to the region of interest may also be used in the assessment of in vivo tissue perfusion. For example, system 1500 may determine whether the particular fluorescence swatch identified as corresponding to the target region corresponds to the particular fluorescence swatch identified as corresponding to the reference region. For example, a target fluorescence swatch and a reference target swatch may correspond to one another if they are the same fluorescence swatch (e.g., if system 1500 determines that fluorescence swatch 1306-5 corresponds to both target region 1310 and to reference region 1402), In other examples, the fluorescence swatches correspond to one another if the fluorescence swatch corresponding to the target region has the same or a greater concentration than the fluorescence swatch that corresponds to the reference region (e.g., if system 1500 determines that fluorescence swatch 1306-6 corresponds to target region 1310 and fluorescence swatch 1306-5 corresponds to reference region 1402). In yet further examples, the fluorescence swatches correspond to one another if the fluorescence swatches are within one fluorescence swatch of one another (e.g., if system 1500 determines that fluorescence swatch 1306-4 corresponds to target region 1310 and fluorescence swatch 1306-5 corresponds to reference region 1402).

System 1500 may be configured to indicate to the user whether the target region has healthy perfusion. For example, if system 1500 determines that the target region does not have healthy perfusion, system 1500 may update the image (e.g., augmented image 1400) to change the pseudo-coloring of the target region to be different from the reference region, display a warning icon or message, and/or perform any other suitable notification operation.

In some examples, a fluorescence evaluation apparatus (e.g., fluorescence evaluation apparatus 100) may be used as an objective reference standard for operational assessment of a fluorescence imaging mode of an imaging device (e.g., imaging device 1202) prior to using the imaging device in a medical procedure. For example, a fluorescence evaluation apparatus may be positioned at a known distance from a distal end of the imaging device system. The fluorescence evaluation apparatus may be manually positioned at the known distance, or system 1500 may be configured to automatically determine the distance, such as based on a 3D depth map, a time-of-flight sensor, and the like. System 1500 may direct the imaging device to illuminate the fluorescence evaluation apparatus with fluorescence excitation illumination and capture an image (e.g., a fluorescence image or an augmented image) of the fluorescence evaluation apparatus.

Based on the captured image, system 1500 may determine whether the fluorescence emitted by one or more fluorescence swatches and detected by the imaging device corresponds with a predetermined (e.g., known or expected) fluorescence signal. For example, system 1500 may determine whether an intensity level of the detected fluorescence from each fluorescence swatch corresponds to a predetermined intensity level (e.g., when the detected intensity level matches the predetermined intensity level within a predefined tolerance, when the detected intensity level exceeds the predetermined intensity level, etc.). The expected fluorescence signal may be stored as calibration data in local memory (e.g., memory 1502) or a remote computing device (e.g., a remote server).

If system 1500 determines that the detected fluorescence corresponds with the predetermined fluorescence signal, system 1500 may confirm that operation of a fluorescence imaging mode of the imaging device is operating properly. System 1500 may provide a notification to the user (e.g., by way of a display device connected to the imaging device) and/or store operational assessment data indicating confirmation of proper operation of the fluorescence imaging mode. If, however, system 1500 determines that the detected fluorescence does not correspond with the predetermined fluorescence signal, system 1500 may determine that the fluorescence imaging mode of the imaging device is not operating properly. Accordingly, in some examples system 1500 may provide a notification to the user (e.g., by way of a display device connected to the imaging device) and/or store operational assessment data indicating that the fluorescence imaging mode is not operating properly. In additional or alternative examples, system 1500 may disable the fluorescence imaging mode of the imaging device.

In some examples, system 1500 may adjust one or more operating parameters of the imaging device until system 1500 determines that the fluorescence imaging mode is operating properly. For instance, system 1500 may adjust an intensity and/or wavelength of the fluorescence excitation illumination source, adjust a gain applied to the detected fluorescence signal, and/or perform any other suitable operation configured to bring the fluorescence imaging mode into proper operation. In this way, system 1500 may be configured to use the fluorescence evaluation apparatus to calibrate the fluorescence imaging mode of the imaging device.

The fluorescence imaging mode assessment provides an assessment of the imaging device as a whole (e.g., the fluorescence excitation illumination source together with the imaging sensor). In some examples, system 1500 may measure an intensity of the fluorescence excitation illumination (e.g., with a monitor photodiode, etc.) and correct the detected fluorescence signal and/or adjust the fluorescence excitation illumination, as necessary, to measure the fluorescence signal under standard conditions. Accordingly, system 1500 may also measure and determine the sensitivity of the imaging sensor to fluorescence.

In some examples, system 1500 may also be configured to use the fluorescence evaluation apparatus to assess and/or calibrate a visible light imaging mode of the imaging device. For example, system 1500 may measure reflected light intensity from a white surface swatch and/or a black surface swatch of the fluorescence evaluation apparatus. Based on the detected visible light signal, system 1500 may determine whether the visible light operating mode of the imaging device is operating properly. If system 1500 determines that the visible light operating mode is operating properly, system 1500 may provide a notification to the user (e.g., by way of a display device connected to the imaging device) and/or store operational assessment data indicating confirmation of proper operation of the visible light imaging mode. If, however, system 1500 determines that visible light imaging mode of the imaging device is not operating properly, system 1500 may provide a notification to the user (e.g., by way of a display device connected to the imaging device) and/or store operational assessment data indicating that the visible light imaging mode is not operating properly. In additional or alternative examples, system 1500 may disable the visible light imaging mode of the imaging device.

In some examples, system 1500 may adjust one or more operating parameters of the imaging device until system 1500 determines that the visible light imaging mode is operating properly. For instance, system 1500 may adjust an intensity and/or wavelength (or wavelength band) of the visible light illumination, adjust a gain applied to the detected visible light signal, perform white balancing, and/or perform any other suitable operation configured to bring the visible light imaging mode into proper operation. In this way, system 1500 may be configured to use the fluorescence evaluation apparatus to calibrate the visible light imaging mode of the imaging device.

A fluorescence evaluation apparatus (e.g., fluorescence evaluation apparatus 100) may also be used as an objective reference standard for comparison of fluorescence imaging of two or more imaging devices. For example, the same fluorescence evaluation apparatus may be imaged by two or more different imaging devices under the same conditions. System 1500 may determine, based on the fluorescence signal detected by each imaging system, the intensity of each detected fluorescence signal relative to the detected fluorescence signal of the other imaging device(s). System 1500 may then provide (e.g., by way of a display device), information indicating the relative fluorescence intensity of imaging device.

In some examples, the imaging system (e.g., imaging system 1200) is connected to, integrated into, or implemented by a surgical system. For example, the imaging system may be connected to, integrated into, or implemented by a computer-assisted surgical system that utilizes robotic and/or teleoperation technology to perform a surgical procedure (e.g., a minimally invasive surgical procedure).

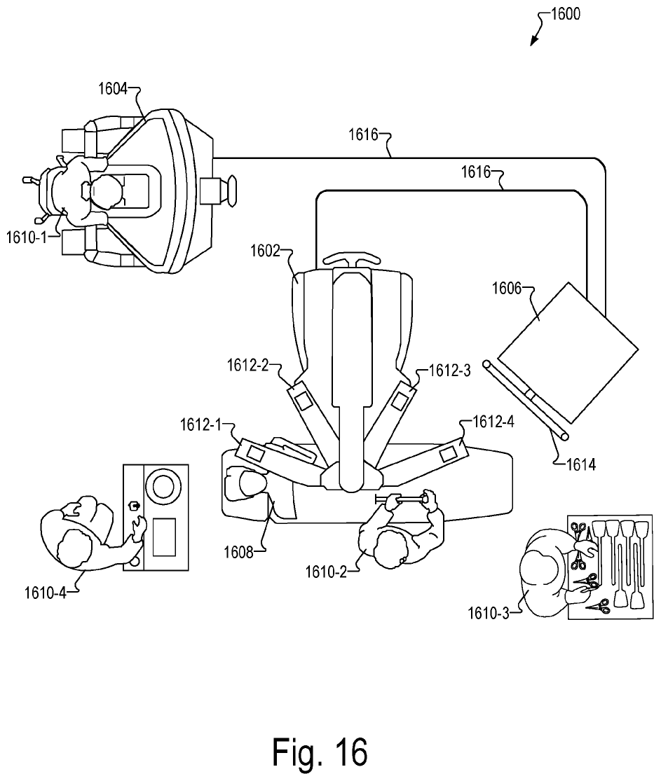

FIG. 16 shows an illustrative computer-assisted surgical system 1600 (surgical system 1600). As shown, surgical system 1600 may include a manipulating system 1602, a user control system 1604, and an auxiliary system 1606 communicatively coupled one to another. Surgical system 1600 may be utilized by a surgical team to perform a computer-assisted surgical procedure on a patient 1608. As shown, the surgical team may include a surgeon 1610-1, an assistant 1610-2, a nurse 1610-3, and an anesthesiologist 1610-4, all of whom may be collectively referred to as surgical team members 1610. Additional or alternative surgical team members may be present during a surgical session as may serve a particular implementation.

While FIG. 16 illustrates an ongoing minimally invasive surgical procedure, it will be understood that surgical system 1600 may similarly be used to perform open surgical procedures or other types of surgical procedures that may similarly benefit from the accuracy and convenience of surgical system 1600. Additionally, it will be understood that the surgical session throughout which surgical system 1600 may be employed might not only include an operative phase of a surgical procedure, as is illustrated in FIG. 16, but may also include preoperative, postoperative, and/or other suitable phases of the surgical procedure. A surgical procedure may include any procedure in which manual and/or instrumental techniques are used on a patient to investigate or treat a physical condition of the patient.

As shown in FIG. 16, manipulating system 1602 may include a plurality of manipulator arms 1612 (e.g., manipulator arms 1612-1 through 1612-4) to which a plurality of surgical instruments may be coupled. Each surgical instrument may be implemented by any suitable surgical tool (e.g., a tool having tissue-interaction functions), medical tool, imaging device (e.g., an endoscope), sensing instrument (e.g., a force-sensing surgical instrument), diagnostic instrument, or the like that may be used for a computer-assisted surgical procedure on patient 1608 (e.g., by being at least partially inserted into patient 1608 and manipulated to perform a computer-assisted surgical procedure on patient 1608). While manipulating system 1602 is depicted and described herein as including four manipulator arms 1612, it will be recognized that manipulating system 1602 may include only a single manipulator arm 1612 or any other number of manipulator arms as may serve a particular implementation.

In some examples, a surgical instrument connected to a manipulator arm 1612 may grasp a fluorescence evaluation apparatus (e.g., fluorescence evaluation apparatus 100) and insert the fluorescence evaluation apparatus into a patient, such as by way of a cannula. Additionally, an imaging device (e.g., imaging device 1202) connected to another manipulator arm may be inserted into the patient and may capture an image of the fluorescence evaluation apparatus at a scene inside the patient.

Manipulator arms 1612 and/or surgical instruments attached to manipulator arms 1612 may include one or more displacement transducers, orientational sensors, and/or positional sensors used to generate raw (e.g., uncorrected) kinematics information. One or more components of surgical system 1600 may be configured to use the kinematics information to track (e.g., determine positions and orientations of) and/or control the surgical instruments.

User control system 1604 may be configured to facilitate control by surgeon 1610-1 of manipulator arms 1612 and surgical instruments attached to manipulator arms 1612. For example, surgeon 1610-1 may interact with user control system 1604 to remotely move or manipulate manipulator arms 1612 and the surgical instruments. To this end, user control system 1604 may provide surgeon 1610-1 with images (e.g., high-definition 3D images, augmented medical images (e.g., augmented images 1300 or 1400), etc.) of a surgical area associated with patient 1608 as captured by an imaging system (e.g., imaging system 1200). In certain examples, user control system 1604 may include a stereo viewer having two displays where stereoscopic images of a surgical area associated with patient 1608 and generated by a stereoscopic imaging system may be viewed by surgeon 1610-1. Surgeon 1610-1 may utilize the images to perform one or more procedures with one or more surgical instruments attached to manipulator arms 1612.

To facilitate control of surgical instruments, user control system 1604 may include a set of master controls. These master controls may be manipulated by surgeon 1610-1 to control movement of surgical instruments (e.g., by utilizing robotic and/or teleoperation technology). The master controls may be configured to detect a wide variety of hand, wrist, and finger movements by surgeon 1610-1. In this manner, surgeon 1610-1 may intuitively perform a procedure using one or more surgical instruments.

Auxiliary system 1606 may include one or more computing devices configured to perform primary processing operations of surgical system 1600. In such configurations, the one or more computing devices included in auxiliary system 1606 may control and/or coordinate operations performed by various other components (e.g., manipulating system 1602 and user control system 1604) of surgical system 1600. For example, a computing device included in user control system 1604 may transmit instructions to manipulating system 1602 by way of the one or more computing devices included in auxiliary system 1606. As another example, auxiliary system 1606 may receive, from manipulating system 1602, and process image data representative of images captured by an imaging device attached to one of manipulator arms 1612.

In some examples, auxiliary system 1606 may be configured to present visual content to surgical team members 1610 who might not have access to the images provided to surgeon 1610-1 at user control system 1604. To this end, auxiliary system 1606 may include a display monitor 1614 configured to display one or more user interfaces, such as images (e.g., 2D images, composite medical images, etc.) of the surgical area, information associated with patient 1608 and/or the surgical procedure, and/or any other visual content as may serve a particular implementation. For example, display monitor 1614 may display images of the surgical area together with additional content (e.g., graphical content, contextual information, etc.) concurrently displayed with the images. In some embodiments, display monitor 1614 is implemented by a touchscreen display with which surgical team members 1610 may interact (e.g., by way of touch gestures) to provide user input to surgical system 1600.

Manipulating system 1602, user control system 1604, and auxiliary system 1606 may be communicatively coupled one to another in any suitable manner. For example, as shown in FIG. 16, manipulating system 1602, user control system 1604, and auxiliary system 1606 may be communicatively coupled by way of control lines 1616, which may represent any wired or wireless communication link as may serve a particular implementation. To this end, manipulating system 1602, user control system 1604, and auxiliary system 1606 may each include one or more wired or wireless communication interfaces, such as one or more local area network interfaces, Wi-Fi network interfaces, cellular interfaces, etc.

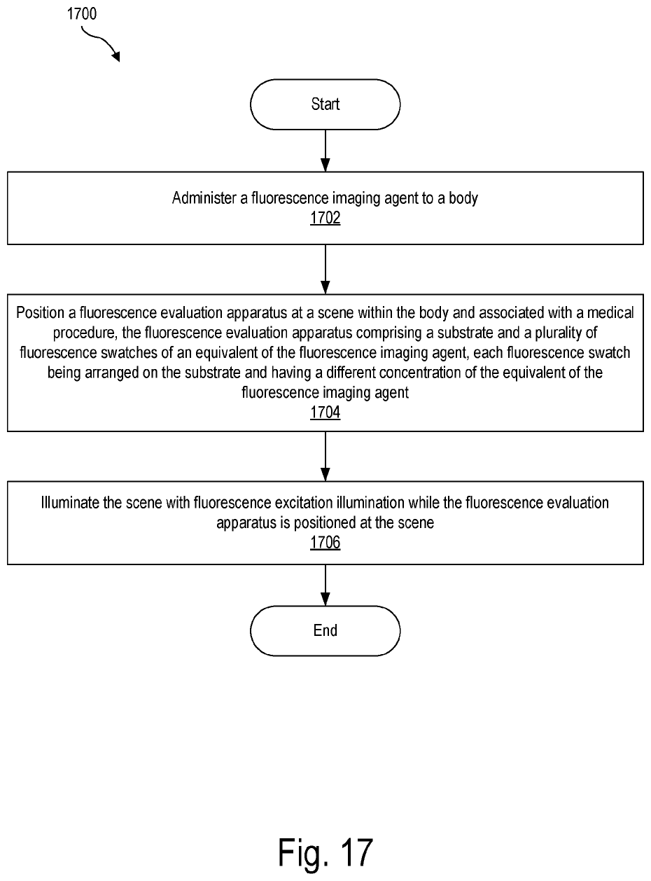

FIG. 17 shows an illustrative method 1700. While FIG. 17 shows steps according to one embodiment, other embodiments may omit, add to, reorder, and/or modify any of the steps shown in FIG. 17. One or more of the steps shown in FIG. 17 may be performed by a user (e.g., a surgical team member 1610) and/or by system 1500, any components included therein, and/or any implementation thereof.

In step 1702, a fluorescence imaging agent is administered to a body. Step 1702 may be performed in any suitable way. For example, the fluorescence imaging agent (e.g., ICG) may be administered to a patient intravenously.

In step 1704, a fluorescence evaluation apparatus (e.g., fluorescence evaluation apparatus 100) is positioned at a scene within the body and associated with a medical procedure (e.g., fluorescence-guided medical procedure, a surgical procedure, etc.). The fluorescence evaluation apparatus comprise a substrate and a plurality of fluorescence swatches of an equivalent of the fluorescence imaging agent, each fluorescence swatch being arranged on the substrate and having a different concentration of the equivalent of the fluorescence imaging agent. Step 1704 may be performed in any suitable way, including any of the ways described herein. For example, the fluorescence evaluation apparatus may be inserted into the body through a cannula, Additionally, the fluorescence evaluation apparatus may be positioned at the scene with a surgical instrument coupled to a computer-assisted surgical system (e.g., a grasping instrument coupled to a manipulator arm 1612 of surgical system 1600). A user may operate a user control system (e.g., manipulate a set of master controls of user control system 1604) to control movement of the surgical instrument and thereby position the fluorescence evaluation apparatus at the scene.

In step 1706, the scene may be illuminated with fluorescence excitation illumination while the fluorescence evaluation apparatus is positioned at the scene. Step 1706 may be performed in any suitable way, including any of the ways described herein. For example, the user may activate a fluorescence imaging mode of an imaging system (e.g., imaging system 1200) to illuminate the scene with fluorescence excitation illumination. Additionally or alternatively, the user may control a position of an imaging device (e.g., an endoscope) at the scene to direct fluorescence excitation illumination emitted by the imaging device to the fluorescence evaluation apparatus and tissue in which the fluorescence imaging agent is present.

In an illustrative implementation of method 1700, the positioning of the fluorescence evaluation apparatus at the scene may comprise positioning the fluorescence evaluation apparatus near a first region of tissue on a first side of an anastomosis at the scene. In implementation of method 1700, a user (e.g., a surgeon) may use the fluorescence evaluation apparatus to visually assess the concentration of the fluorescence imaging agent present at the first region of tissue. In some examples, the fluorescence evaluation apparatus may also be repositioned near a second region of tissue on a second side of the anastomosis at the scene. In this way, the user may use the fluorescence evaluation apparatus to visually assess perfusion of the first region of tissue and/or the second region of tissue.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Suitable non-volatile storage media include, but are not limited to, read-only memory, flash memory, a solid-state drive, a magnetic storage device (e.g. a hard disk, a floppy disk, magnetic tape, etc.), ferroelectric random-access memory (RAM), and an optical disc (e.g., a compact disc, a digital video disc, a Blu-ray disc, etc.). Suitable volatile storage media include, but are not limited to, RAM (e.g., dynamic RAM).

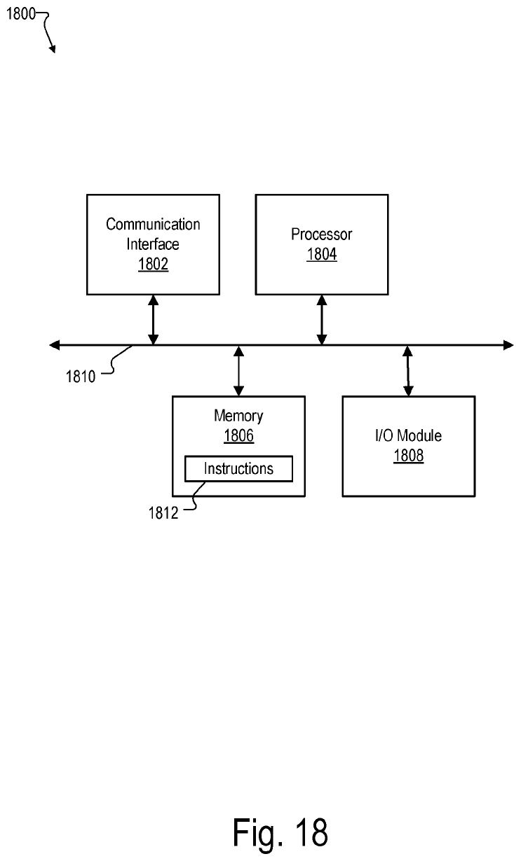

FIG. 18 shows an illustrative computing device 1800 that may be specifically configured to perform one or more of the processes described herein. As shown in 18, computing device 1800 may include a communication interface 1802, a processor 1804, a memory 1806, and an input/output (I/O) module 1808 communicatively connected one to another via a communication infrastructure 1810. While an illustrative computing device 1800 is shown in FIG. 18, the components illustrated in FIG. 18 are not intended to be limiting. Additional or alternative components may be used in other embodiments. Components of computing device 1800 shown in FIG. 18 will now be described in additional detail.

Communication interface 1802 may be configured to communicate with one or more computing devices. Examples of communication interface 1802 include, without limitation, a wired network interface (such as a network interface card), a wireless network interface (such as a wireless network interface card), a modem, an audio/video connection, and any other suitable interface.

Processor 1804 generally represents any type or form of processing unit capable of processing data and/or interpreting, executing, and/or directing execution of one or more of the instructions, processes, and/or operations described herein. Processor 1804 may perform operations by executing computer-executable instructions 1812 (e.g., an application, software, code, and/or other executable data instance) stored in memory 1806.

Memory 1806 may include one or more data storage media, devices, or configurations and may employ any type, form, and combination of data storage media and/or device. For example, memory 1806 may include, but is not limited to, any combination of the non-volatile media and/or volatile media described herein. Electronic data, including data described herein, may be temporarily and/or permanently stored in memory 1806. For example, data representative of computer-executable instructions 1812 configured to direct processor 1804 to perform any of the operations described herein may be stored within memory 1806. In some examples, data may be arranged in one or more databases residing within memory 1806.

I/O module 1808 may include one or more I/O modules configured to receive user input and provide user output. One or more I/O modules may be used to receive input for a single virtual experience. I/O module 1808 may include any hardware, firmware, software, or combination thereof supportive of input and output capabilities. For example, I/O module 1808 may include hardware and/or software for capturing user input, including, but not limited to, a keyboard or keypad, a touchscreen component (e.g., touchscreen display), a receiver (e.g., an RF or IR receiver), motion sensors, and/or one or more input buttons.

I/O module 1808 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1808 is configured to provide graphical data to a display for presentation to a user. The graphical data may be representative of one or more graphical user interfaces and/or any other graphical content as may serve a particular implementation.

In some examples, any of the systems, computing devices, and/or other components described herein may be implemented by computing device 1800. For example, processor 1504 may be implemented by processor 1804 and memory 1502 may be implemented by memory 1806.

1-55. (canceled)

56. A fluorescence evaluation apparatus comprising:
a substrate configured to be inserted into a body through a channel having an inside diameter between about 5 millimeters (mm) and about 30 mm; and
a plurality of fluorescence swatches of a fluorescence imaging agent, each fluorescence swatch being arranged on the substrate and having a different concentration of the fluorescence imaging agent.

57. The fluorescence evaluation apparatus of claim 56, wherein the plurality of fluorescence swatches are arranged on the substrate in an order of increasing concentration of the fluorescence imaging agent.

58. The fluorescence evaluation apparatus of claim 56, wherein:
a first fluorescence swatch of the plurality of fluorescence swatches has a first concentration of the fluorescence imaging agent;
a second fluorescence swatch of the plurality of fluorescence swatches has the first concentration of the fluorescence imaging agent; and
the second fluorescence swatch is separated from the first fluorescence swatch by one or more additional fluorescence swatches of the plurality of fluorescence swatches positioned between the first fluorescence swatch and the second fluorescence swatch.

59. The fluorescence evaluation apparatus of claim 56, wherein the fluorescence imaging agent comprises one or more of indocyanine green (ICG), IR-125, or quantum dots.

60. The fluorescence evaluation apparatus of claim 56, wherein:
the fluorescence imaging agent is configured to be excited by fluorescence excitation illumination having a wavelength between about 750 nm and about 810 nm; and
the fluorescence imaging agent is configured to emit, in response to excitation by the fluorescence excitation illumination, fluorescence having a peak intensity at a wavelength between about 800 nm to about 850 nm.

61. The fluorescence evaluation apparatus of claim 56, wherein the substrate is substantially transparent to near-infrared light.

62. The fluorescence evaluation apparatus of claim 56, wherein each fluorescence swatch of the plurality of fluorescence swatches comprises surface irregularities.

63. The fluorescence evaluation apparatus of claim 56, wherein each fluorescence swatch of the plurality of fluorescence swatches comprises an optical dust from an optical filter material configured to filter light having a wavelength that is shorter than a peak wavelength of fluorescence emitted by the fluorescence imaging agent.

64. The fluorescence evaluation apparatus of claim 56, further comprising distance scale markers arranged on the substrate, wherein the distance scale markers comprise the fluorescence imaging agent.

65. The fluorescence evaluation apparatus of claim 56, further comprising a near-infrared (NIR) light swatch arranged on the substrate and comprising an up-converting fluorescence imaging agent configured to emit visible light when excited by NIR light.

66. A fluorescence evaluation apparatus comprising:
a substrate;
a plurality of fluorescence swatches of a fluorescence imaging agent, each fluorescence swatch being arranged on the substrate and comprising a different concentration of the fluorescence imaging agent; and
a quenching element configured to quench specular reflection, from the plurality of fluorescence swatches, of light having a wavelength that is shorter than a peak wavelength of fluorescence emitted by the fluorescence imaging agent.

67. The fluorescence evaluation apparatus of claim 66, wherein the quenching element comprises an optical dust, from an optical filter material, mixed with the fluorescence imaging agent, the optical filter material configured to filter light having the wavelength that is shorter than the peak wavelength of fluorescence emitted by the fluorescence imaging agent.

68. The fluorescence evaluation apparatus of claim 66, wherein the quenching element comprises a layer of optical filter material over the plurality of fluorescence swatches, the optical filter material configured to filter light having the wavelength that is shorter than the peak wavelength of fluorescence emitted by the fluorescence imaging agent.

69. The fluorescence evaluation apparatus of claim 66, wherein the quenching element comprises surface irregularities on surfaces of the plurality of fluorescence swatches.

70. A method comprising:
forming a substrate configured to be inserted into a body through a channel having an inside diameter between about 5 millimeters (mm) and about 30 mm; and
arranging, on the substrate, a plurality of fluorescence swatches of a fluorescence imaging agent, each fluorescence swatch having a different concentration of the fluorescence imaging agent.

71. The method of claim 70, further comprising forming a plurality of fluorescence tiles comprising the fluorescence imaging agent, each fluorescence tile having a different concentration of the fluorescence imaging agent;
wherein the forming the substrate comprises forming a plurality of recessed wells in each of a plurality of distinct regions of the substrate; and
wherein the arranging the plurality of fluorescence swatches on the substrate comprises securing the plurality of fluorescence tiles in the plurality of recessed wells.

72. The method of claim 71, wherein the forming the plurality of fluorescence tiles includes mixing a base material having a non-white color with the fluorescence imaging agent.

73. The method of claim 71, wherein the forming the plurality of fluorescence tiles includes:
mixing, with the fluorescence imaging agent, an optical dust from an optical filter material, the optical filter material configured to filter light having a wavelength that is shorter than a peak wavelength of fluorescence emitted by the fluorescence imaging agent.

74. The method of claim 70, further comprising sanding the plurality of fluorescence swatches to form surface irregularities on surfaces of the plurality of fluorescence swatches.

75. The method of claim 70, further comprising applying a layer of optical filter material over the plurality of fluorescence swatches, the optical filter material configured to filter light having a wavelength that is shorter than a peak wavelength of fluorescence emitted by the fluorescence imaging agent.
